SEQUENCE LISTING (8 sequences)

SEQ ID NO: 1 - DNA, 20 bp, Artificial Sequence
  Forward primer for IL-8 locus
agctgcagaaatcaggaagg

SEQ ID NO: 2 - DNA, 20 bp, Artificial Sequence
  Reverse primer for IL-8 locus
tataaaaagccaccggagca

SEQ ID NO: 3 - DNA, 20 bp, Artificial Sequence
  Targeting IL-8
ttcagtgtaaagctttctga

SEQ ID NO: 4 - DNA, 20 bp, Artificial Sequence
  Targeting Cxcl15
acagagcagtcccaaaaaat

SEQ ID NO: 5 - DNA, 25 bp, Artificial Sequence
  Forward primer for IL-8
tttggacttagactttatgcctgac

SEQ ID NO: 6 - DNA, 21 bp, Artificial Sequence
  Reverse primer for IL-8
tcctgggcaaactatgtatgg

SEQ ID NO: 7 - DNA, 26 bp, Artificial Sequence
  Forward primer for Cxcl 15
gctaggcacactggatatgtgttaaa

SEQ ID NO: 8 - DNA, 20 bp, Artificial Sequence
  Reverse primer for Cxcl 15
acatttggggatgctactgg

(19) United States
(12) Patent Application Publication
(10) Pub. No.: US 2022/0040252 A1
(43) Pub. Date: Feb. 10, 2022

(54) TREATMENT OF PROSTATE CANCER BY ANDROGEN ABLATION AND IL-8 BLOCKADE

(71) Applicant: The Trustees of Columbia University in the City of New York, New York, NY (US)

(72) Inventors: Charles G. Drake, New York, NY (US); Zoila Areli Lopez-Bujanda, Bethesda, MD (US)

(21) Appl. No.: 17/407,537

(22) Filed: Aug. 20, 2021

Related U.S. Application Data
(63) Continuation of application No. PCT/US2020/018765, filed on Feb. 19, 2020.
(60) Provisional application No. 62/809,060, filed on Feb. 22, 2019.

(51) Int. Cl. A61K 38/09; A61K 31/58; A61K 31/167; A61K 31/277; A61K 31/4166; A61K 31/4439; A61K 39/395; A61P 35/00

(52) U.S. Cl. CPC A61K 38/09; A61K 31/58; A61K 31/167; A61P 35/00; A61K 31/4166; A61K 31/4439; A61K 39/3955; A61K 31/277

(57) ABSTRACT

A method of treating prostate cancer by administration of an IL-8 blocker in combination with androgen ablation.

### REFERENCE TO SEQUENCE LISTING SUBMITTED BY EFS-WEB

The contents of the ASCII text file of the sequence listing named 8441-0021WO-ST25, which is 1.91 kb in size, was created on Feb. 18, 2020, and was electronically submitted via EFS-Web with this application, is incorporated herein by reference in its entirety.

### REFERENCE TO RELATED APPLICATIONS

This application is a continuation of International Application No. PCT/US2020/018765 filed on Feb. 19, 2020, which claims the benefit of United States provisional patent application 62/809060 filed Feb. 22, 2019, each of which is hereby incorporated herein in its entirety for all purposes as if fully set forth herein.

### GOVERNMENT RIGHTS

This invention was made with government support under W81XWH-13-1-0369 awarded by Army/MRMC. The government has certain rights in the invention.

### FIELD OF THE INVENTION

The present invention relates to methods of treatment for prostate cancer by administration of an IL-8 antagonist or an IL-8 receptor antagonist in combination with androgen ablation and optionally in combination with other therapies such as chemotherapy, radiotherapy, or immunotherapy.

### BACKGROUND OF THE INVENTION

The following discussion is provided merely to aid the reader in understanding the disclosure and is not admitted to describe or constitute prior art thereto.

Prostate cancer is the most commonly diagnosed and third deadliest malignancy among men in the United States. It is estimated that one in seven American men will receive a diagnosis of prostate cancer at some point in their lives, at an average age of 68 years. In 2017, there were over 160,000 newly diagnosed cases and 26,000 deaths from prostate cancer in the United States alone. Patients with localized disease are typically treated surgically or with radiation therapy. However, 20-40% of patients undergoing a radical prostatectomy and 30-50% of patients receiving radiation therapy will have recurrence of disease. Standard therapy for metastatic disease generally involves androgen ablation, either by bilateral orchiectomy or androgen deprivation therapy (ADT). Although androgen ablation is highly effective, patients eventually develop castration-resistant prostate cancer (CRPC). A number of therapeutic agents have been approved by the FDA for treatment of CRPC and have shown positive impact, but metastatic CRPC currently has no curative treatment option. Several investigators have reported that, without treatment, median survival time ranges from 9.1 to 21.7 months. Recent studies have suggested that infiltration of myeloid-derived suppressor cells (MDSC) into prostate tumor is related to the failure of androgen ablation. It seems likely that the immunosuppressive environment established by MDSCs hinders the antitumor response in prostate cancer. Any therapy that would improve prospects for these patients would be a significant advance in prostate cancer treatment.

### SUMMARY OF THE INVENTION

Provided herein is a method of treatment of prostate cancer in a patient in need of such treatment which comprises administering to the patient an effective amount of an IL-8 antagonist or an IL-8 receptor antagonist (collectively sometimes described herein as “IL-8 blockers”) in combination with androgen ablation.

The inventors have surprisingly discovered that androgen ablation (such as ADT) results in the secretion of IL-8 from prostate epithelial cells. IL-8 is a potent chemoattractant that recruits and maintains immune cells with suppressive properties, such as myeloid derived suppressor cells (MDSCs). ADT results in an influx of PMN-MDSC into prostate tumors. Blocking the IL-8/IL-8 receptor (CXCR1 and CXCR2) interaction leads to a decrease in the infiltration of suppressive immune cells into prostate tumors and is expected to lead to improved clinical results.

In one aspect of the invention, disclosed herein is a method of treating prostate cancer in a patient in need of such treatment which comprises administering to said patient in combination with androgen ablation an amount of a compound selected from an IL-8 antagonist and an IL-8 receptor antagonist, the amount of said compound being effective to at least reduce the level of infiltration of suppressive immune cells into prostate tumor. The level of infiltration may be determined (for example) in pre- and post- treatment biopsy specimens by protein or RNA quantification using, but not limited to, at least one of the following methodologies: IF, IHC, flow cytometry, ELISA, CyTOF, CITE-Seq, PCR, RISH, RNAseq, or nanostring.

A number of IL-8 blockers have been or are currently being clinically evaluated for treatment of a variety of diseases, including arthritis, COPD, psoriasis, inflammatory disorders, and breast cancer. None of these IL-8 blockers has been suggested for possible use in treating prostate cancer in combination with androgen ablation. These compounds include BMS-986253 (HuMax IL-8; Bristol-Myers Squibb), ABX-IL8, HuMab 10F8, SCH527123/MK-7123, AZD5069, AZD5122, AZD8304, RIST4721 (AstraZeneca), CCX832 (ChemoCentrix/GlaxoSmithKline), a CX3CR1 antagonist NEUROCRINE (Neurocrine Biosciences), a CXCR1/2 monoclonal IL-8 blocker (Eli Lilly), a CXCR2 antagonist CHEMOCENTRIX (ChemoCentrix), CXCR2 antagonists ASTRAZENECA (AstraZeneca), CXCR2 biparatopic nanobodies ABLYNX (Ablynx/Novartis), a CXCR2 monoclonal IL-8 blocker PEPSCAN (Pepscan/Medimmune, AstraZeneca), DF1970, DF2726A, DF2156A, DF2162, DF2755A, repertaxin, reparixin (Dompe), FX68 (Janssen), GSK1325756, GSK1325756H, SB225002, SB251353, SB332235, SB656933 (GlaxoSmithKline), KB03 (Kerbos), MGTA145 (Magenta), PACG31P (Pacgen Life Science), PS291822 (navarixin; Pharmacopeia), SX576, SX682 (Syntrix Biosystems), and other human ELR+CXC chemokine blockers. Many of the compounds have reached Phase II testing and one (reparixin) has reached phase III testing. Based on the available information regarding these IL-8 blockers, one of skill in the art would readily understand how to use these compounds in the practice of the disclosed invention.

In one aspect, the present disclosure provides methods for treating prostate cancer in a patient in need of such treatment by administering to said patient a therapeutically effective amount of at least one IL-8 blocker in combination with androgen ablation, wherein the IL-8 blocker is selected from BMS-986253 (HuMax IL-8), ABX-IL8, HuMab 10F8, SCH527123/MK-7123, AZD5069, AZD5122, AZD8304, RIST4721, CCX832, a CX3CR1 antagonist, a CXCR1/2 monoclonal IL-8 blocker, a CXCR2 antagonist, CXCR2 biparatopic nanobodies, a CXCR2 monoclonal IL-8 blocker, DF1970, DF2726A, DF2156A, DF2162, DF2755A, repertaxin, reparixin, FX68, GSK1325756, GSK1325756H, SB225002, SB251353, SB332235, SB656933, KB03, MGTA145, PACG31P, PS291822 (navarixin), SX576, SX682, and other human ELR+CXC chemokine blockers.

Androgen ablation may be performed by bilateral orchiectomy or by administration of Androgen Deprivation Therapy (ADT). ADT may be performed by administration of a compound such as, for example, an LHRH agonist (e.g., leuprolide, goserelin, triptorelin, or histrelin); an LHRH antagonist (e.g., degarelix, a CYP17 inhibitor, or abiraterone); a drug to stop androgen function, such as an anti-androgen (e.g., flutamide, bicalutamide, nilutamide, enzalutamide, or apalutamide); an estrogen; and ketoconazole.

In another aspect, the present disclosure provides methods for treating prostate cancer in a human in need of such treatment by administering to said patient a therapeutically effective dose of at least one IL-8 blocker in combination with androgen deprivation therapy (ADT). The IL-8 blocker may be selected from BMS-986253 (HuMax IL-8), ABX-IL8, HuMab 10F8, SCH527123/MK-7123, AZD5069, AZD5122, AZD8304, RIST4721, CCX832, a CX3CR1 antagonist, a CXCR1/2 monoclonal IL-8 blocker, a CXCR2 antagonist, CXCR2 biparatopic nanobodies, a CXCR2 monoclonal IL-8 blocker, DF1970, DF2726A, DF2156A, DF2162, DF2755A, repertaxin, reparixin, FX68, GSK1325756, GSK1325756H, SB225002, SB251353, SB332235, SB656933, KB03, MGTA145, PACG31P, PS291822 (navarixin), SX576, SX682, and other human ELR+CXC chemokine blockers. The IL-8 blocker may be administered before, concurrently with, or after the androgen deprivation therapy, but administration of the IL-8 blocker before ADT is preferred.

Other therapies may be used in combination with the disclosed method. Such other therapies include radiotherapy, immunotherapy, and chemotherapy, but immunotherapy is preferred. Preferred immunotherapy agents are anti-PD-1, anti-PD-L1, anti-CTLA-4, anti-TIM-3, anti-TGIT, anti-CD40, a TLR agonist, a STING agonist, bi-specific T cell engagers (BiTEs) and dual-affinity retargeting antibodies (DARTs), chimeric antigen receptors (CARs) T cells, and anti-cancer vaccines such as Sipuleucel-T, PSA-TRICOM, AVAX Tech vaccines, Prostvac-VF, and a listeria-based vaccines such as live attenuated double-deleted (LADD) and ADXS031-142.

In another aspect, the present disclosure provides methods for treating prostate cancer in a patient in need of such treatment by administering to the patient a therapeutically effective amount of at least one IL-8 blocker in combination with androgen deprivation therapy and immunotherapy, wherein the immunotherapeutic agent is selected from anti-PD-1, anti-PD-L1, anti-CTLA-4, anti TIM-3, anti-TGIT, anti-CD40, a TLR agonist, a STING agonist, bi-specific T cell engagers (BiTEs) and dual-affinity retargeting antibodies (DARTs), chimeric antigen receptors (CARs) T cells, and an anti-cancer vaccine. The anti-cancer vaccine may be, for example, Sipuleucel-T, PSA-TRICOM, AVAX Tech vaccines, Prostvac-VF, or a listeria-based vaccine such as live attenuated double-deleted (LADD) and ADXS031-142. The three therapies (IL-8 blockade, ADT, and immunotherapy) may be performed in any order, but the preferred order is to administer immunotherapy and IL-8 blockade before ADT.

As used herein, the term “IL-8 blocker” includes antagonists to IL-8 (CXCL8), and antagonists to the IL-8 receptors (CXCR1 and CXCR2). The term “IL-8 blockade” means interfering with, decreasing, or entirely blocking the interaction between IL-8 and its receptor(s). IL-8 blockers useful in the claimed method include those materials referenced herein but is not limited thereto. It is expected that any IL-8 blocker discovered in the future would be useful in the present method.

In some embodiments, the IL-8 blocker is administered orally at doses between 50 to 1,200 mg (1 to 3 times a day) per kg for up to 2 years or by intravenous infusions at doses between 1 to 50 mg (every 1 to 4 weeks) per kg. Preferred doses for iv administration may range from 5 mg/kg to 40 mg/kg. For example, the iv dose of BMS-986253 may be 4 mg/kg, 8 mg/kg, 16 mg/kg, or 32 mg/kg. It is expected that a skilled practitioner in the cancer treatment field could readily determine an appropriate dosage and regimen.

In another aspect, the present disclosure provides methods of treating a patient with prostate cancer comprising administering in combination with androgen ablation a therapeutically effective amount of an IL-8 blocker to a patient in need thereof 1 to 3 times a day if administered orally or every 1 to 4 weeks if administered intravenously.

The foregoing general description and following detailed description are exemplary and explanatory and are intended to provide further explanation of the invention.

### BRIEF DESCRIPTION OF THE DRAWINGS

FIG. 1 shows that androgen deprivation therapy (ADT) increases IL-8 transcription in prostate cancer. a, Androgen responsive tumor epithelial cells progress from castration-sensitive (CS) to androgen responsive (ADT), and eventually developed castration-resistance (CR). CR was tumor size defined as 30% of nadir tumor volume. Left, fluorescent tagin strategy to generate mCherry⁺ Myc-Cap cells (MCRedAL cells). Right, tumor growth curve of MCRedAL tumors. CTX: Castration (n≥3 per group, repeated×2). b, Sorting strategy to isolate tumor epithelial cells from a based on their expression of mCherry and their CD45⁻CD11b⁻F4/80⁻ phenotype. c, Differential expression profile of tumor epithelial cells isolated from castration-sensitive (CS) and ADT-treated MCRedAL tumor bearing mice. Heatmap showing transcripts 3 standard deviations away from the mean (n=3 per group). d, Differential chemokine expression of tumor epithelial cells isolated from CS and ADT tumor bearing mice (replicate numbers as in c). Left, volcano plot showing gene expression among all MTA 1.0 microarray transcripts. Right, heatmap of normalizedchemokine transcripts. e, Hallmarks gene sets pathway analysis post-ADT shows NF-κB up-regulation post-ADT. f, qRT-PCR quantification of IL-8 in LNCaP cells cultured at indicated concentrations of TNFα and DHT, cells cultured in androgen-free media as described in materials and methods (n=3 per condition, repeated×2). Expression levels normalized to mean ΔCT level in samples cultured in androgen free media without TNFα or DHT. g, ChIP-Seq analysis of AR at the IL-8 (CXCL8) promoter in LNCaP cells cultured in the presence of either vehicle (DMSO), DHT (100 nM), or TNFα (1000 U/ml) (n=2 per group; GSE83860). h, ChIP quantitative RT-PCR (qRT-PCR) analysis of AR, pSer2 Pol II, pol II, and H3K4me3 at the IL-8 (CXCL8) and PSA (KLK3) promoter, left and right respectively (n=3 per group). Transfected LNCaP cells treated for 24 hours with or without DHT (100 nM). For e, loci with significant differential binding (black bar) were identified as described in materials and methods. Error bars represent standard error. Unpaired t-tests were performed, p-values≤0.05 (*), 0.01 (**), 0.001 (***) and 0.0001 (****); p-values 0.05 (ns).

FIG. 2 shows that IL-8 is differentially expressed in castration-resistant versus castration-sensitive prostate cancer cells. a, Representative images of Cxcl15 fluorescent detection (murine homologue of IL-8) in Myc-Cap tumors. Tumors were harvested when volumes reached ˜500 mm3 (CS group), 7 days after androgen-deprivation (ADT), or at the time of castration-resistance (CR) and hybridized with CF568-labeled probe sets (white) to Cxcl15, CF640-labeled anti-PanCK antibody (red), and CF488-labeled anti-CD45 antibody (green). Nuclei counterstained with DAPI (blue). Repeated×3. b, Gene and protein expression of Cxcl15 in MCRedAL cells of indicated tumor samples by qRT-PCR and ELISA, respectively (n=3 per group, repeated×2). c, qRT-PCR quantification of IL-8 in human AR positive castration-sensitive cells (CS: LNCaP, LAPC4, and VCaP) and their castration-resistant counterparts (CR: LNCaP-abl, LAPC4-CR, and VCaP-CR), replicate numbers as in b. d, IL-8 protein expression in the isogenic cell pairs from c quantified by ELISA, replicate numbers as in c. e, qRT-PCR quantification of IL-8 in AR positive castration-sensitive (LNCaP, LAPC4, and VCaP) and AR independent castration-resistant (E006AA, CWR22Rv1, DU145, and PC3) human prostate cancer cell lines (n=2 per group, repeated×2). f, Representative images of Cxcl15 fluorescent detection in benign murine prostate tissue samples from castration-sensitive (CS), androgen-deprivation treated (ADT), and ADT-treated mice that received testosterone repletion (ADT+T). Tissue sections hybridized with CF568-labeled probe sets (white) to Cxcl15, and CF640-labeled anti-PanCK antibody (red). Nuclei were counterstained with DAPI (blue). Repeated×3. g, qRT-PCR analysis of Cxcl15 expression in prostate luminal epithelial cells from indicated treatment groups (n=3 per group). Prostate luminal epithelial cells were isolated based on their GFP⁺CD49f^{int}CD24⁺CD45⁻F4/80⁻CD11b⁻ expression by flow sorting into Trizol LS. h, Expression of IL-8 in human prostate epithelial cells micro-dissected from patients in a clinical trial (NCT00161486) receiving placebo, androgen-deprivation treatment (ADT), or ADT plus testosterone repletion (ADT+T). Z-score values of microarray transcripts from benign prostate biopsies were normalized to placebo samples (N=4 per group; GSE8466). i, Expression of IL-8 in human prostate c 174 ancer epithelial cells micro-dissected from untreated or ADT-treated (NCT01696877; n=8 per group) patients as determined by qRT-PCR. RISH images are at 60× magnification; scale bar=100 μm. Gene expression levels were normalized to the mean ΔCT level in samples from CS, untreated or placebo groups. For b-h, unpaired t-tests were performed; for i a Mann-Whitney U test was used due to the non-normal data distribution observed. p-values≤0.05 (*) and 0.01 (**); p-values≥0.05 (ns) shown. The range in box and whiskers plots shows min and max values such that all data are included.

FIG. 3 shows that ADT-driven IL-8 (and Cxcl15) up-regulation promotes PMN-MDSC infiltration. a, Gating strategy used to profile the immune compartment of the TME by flow cytometry. Tumor associated macrophages (TAMs) gated based on CD45⁺Ly6G⁻F4/80⁺CD11 b⁺, Inflammatory (Inf.) TAMs as CD45⁺CD11b⁺F4/80⁺Ly6C⁺MHCII⁻, immature (Imm.) TAMs as CD45⁺CD11b⁺F4/80⁺Ly6C⁺MHCII⁺, MHCII^{hi} TAMs as CD45⁺CD11b⁺F4/80⁺Ly6C⁻MHCII⁺, MHCII^{low} TAMs as CD45⁺CD11b⁺F4/80⁺Ly6C⁻MHCII⁻, tumor Infiltrating Lymphocytes (TILs) CD45⁺CD4⁺ or CD45⁺CD8⁺, tumor infiltrating polymorphonuclear myeloid-derived suppressor cells (PMN-MDSCs) as CD45⁺CD11b⁺Ly6C⁺Ly6G⁺. b, TAM, TIL, and PMN-MDSC density normalized to mg of tumor weight (cells/mg; n≥3 per group, repeated×2). c, Representative H&E and immunohistochemistry (F4/80 and Ly6G) of indicated murine allografts (repeated×3). d, Normalized expression of selected genes determined by NanoString nCounter gene analysis in sorted myeloid fractions defined as in a (n=3 per group). e, qRT-PCR quantification of Cxcr2 and II-23 in indicated populations of Myc-Cap tumors (n=3 per group). f, Representative histograms of protein expression determined by flow cytometry in PMN-MDSCs from indicated organs (repeated×2). g and h, Density of PMN-MDSCs normalized to mg of tumor weight (cells/mg) in Myc-Cap and PC3 tumors (n≥4 per group, repeated×2). Cells quantified by flow cytometry as in a, tumors implanted and harvested as described below. H&E and IHC images at 40× magnification; scale bar=50 pm. Gene expression levels normalized to the mean ACT level in samples from the Immature TAMs (Imm.) group. Unpaired t-tests performed, p-values≤0.05 (*), 0.01 (**), 0.001 (***) and 0.0001 (****); p-values≥0.05 (ns).

FIG. 4 shows that blockade of the CXCR2/IL8 pathway attenuates the migration of PMN-MDSCs but not their function. a, Analysis of Ly6G+ PMNs in peritoneal washings receiving Cxcl15 (200 ng/mouse, i.p.) in mice pre-treated with either isotype or αCXCR2 (n≥4 per group, repeated×2). b, Analysis of the fold change between the number of Ly6G⁺ PMNs in peritoneal washings from a in relation to PMNs' numbers in peripheral blood of indicated treated mice. c, Representative plots of Ly6G⁺ PMNs in peritoneal washings from a of indicated treated mice (repeated×2). d, PMN-MDSC in vitro migration towards tumor supernatants in the presence of either isotype or anti-CXCR2 (200 μg/ml). Antibodies were added at the beginning of the experiment (n≥2 per group, repeated×2). e, PMN-MDSC in vitro migration towards CR-LNCaP (LNCaP-abl) WT or IL-8 KO tumor supernatants (n=3 per group, repeated×2). f, Schematic representation of PMN-MDSC suppression assay. OT-I splenocytes (CD45.2) were mixed with naïve splenocytes (CD45.1) in a 1:10 ratio, labeled with CTV, and co-culture with PMN-MDSCs at the indicated ratios. T cell proliferation was stimulated (Stim) by OVA peptide (5 μM) for 60 hours. g, Percent suppression when either unselected or low-density PMN-MDSCs were used for the experiment (n=3 per group, repeated×3). h, Percent of CD8 T cells (left) and antigen specific OT-I cells (CD45.2; right) proliferating at different proportions of PMN-MDSCs when stimulated with or without 5 μM of OVA, replicate numbers as in g. i, Representative histograms of antigen specific OT-I cells proliferation based on the dilution of CTV dye when stimulated as in h (repeated×2). j, Percent suppression in the presence of either isotype or anti-CXCR2 (200 μg/ml). Antibodies were added at the beginning of the experiment (n=3 per group, repeated×2). k, Percent suppression of PMN-MDSCs derived from spleens of WT or Cxcl15 KO Myc-Cap tumor bearing mice (n=3 per group, repeated×2). For a-c, PMNs were gated on CD45⁺Ly6G⁺ cells. Cell migration in vivo was evaluated 4 hours after PBS or cytokine treatment and normalized to 10,000 beads. PBS was injected as the control for these experiments. For d-e, PMN-MDSCs were isolated from spleens of mice bearing CR-Myc-Cap tumors and placed in the top chamber of a transwell. Culture supernatants were plated in the bottom chamber, and number of PMN-MDSCs migrating from the top to the bottom chamber after 2.5 hours was evaluated. For g, j-k, percent suppression (% Suppression) was calculated by the following formula: % Suppression=[1−(% divided cells of the condition/the average of % divided cells of T responder only conditions)]×100. Unpaired t-tests were performed, p-values≤0.05 (*), 0.01 (**), 0.001 (***) and 0.0001 (****); p-values≥0.05 (ns).

FIG. 5 shows that CXCR2 blockade improves response to checkpoint blockade following androgen-deprivation in a physiologically relevant model of prostate cancer. a, Treatment scheme, scale=weeks. Animals sacrificed for immune phenotyping 1 week post-ADT. b, Tumor growth and survival curves of mice from isotype vs. anti-CTLA-4 vs. anti-CTLA-4+anti-CXCR2 groups treated as described in a (black line vs. orange line vs. purple line, respectively; n≥8 per group, repeated×2). c, Tumor infiltrating lymphocyte (TILs) density in indicated treatment groups (n≥5 per group, repeated×2). d, Treg percentages (as fraction of CD4) in indicated tissues (n≥5 per group, repeated×2). e, Polyfunctional CD8 T cells, left panel=density, center/right panels=percentage of total CD8, animals numbers as in d. f, Representative histograms and dot plots of polyfunctional CD8⁺ IFNγ⁺Gzβ⁺TNFα⁺ from tumor draining lymph nodes (TDLN). Repeated×2. For a-f, treatment was initiated when tumor volumes reached 200 mm³. Average tumor volume (±s.e.m.) for each experimental group. Wilcoxon test used for survival analysis. Flow cytometry as in materials and methods. Unpaired t-tests performed, p-values≤0.05 (*), 0.01 (**), 0.001 (***) and 0.0001 (****); p-values≥0.05 (ns).

FIG. 6 shows that the therapeutic effect of the triple combination associates with a reduction in tumor infiltrating PMN-MDSCs. a, Tumor growth and survival curves of mice from isotype vs. αCXCR2 treatment groups (green vs. blue, respectively; n=10 per group, repeated×2). b, Tumor growth and survival curves of mice from isotype vs. αCTLA-4 vs. αCTLA-4+αCXCR2 treatment groups (green vs. orange vs. purple, respectively; n≥7per group, repeated×2). c, Tumor growth and survival curves of mice from isotype vs. αCSF1R treatment groups (green vs. purple, respectively; n≥7 per group, repeated×2). d, PMN-MDSCs as a percentage of CD45⁺ cells in the TME of indicated treatment groups, replicate numbers as in b. e, TAMs as a percentage of CD45⁺ cells in the TME of indicated treatment groups, replicate numbers as in c. CSF1R treatment groups (green vs. purple, respectively; n≥7 per group, repeated×2). d, PMN-MDSCs as a percentage of CD45⁺ cells in the TME of indicated treatment groups, replicate numbers as in b. e, TAMs as a percentage of CD45⁺ cells in the TME of indicated treatment groups, replicate numbers as in c. f, Memory CD4 T cells as a percentage of CD45⁺CD4⁺ T cells in the tumor (tumor infiltrating lymphocytes: TILs) and tumor-draining lymph node (TDLN) of indicated treatment groups (n≥5 per group, repeated×2). g, Memory CD8 T cells as a percentage of CD45⁺CD8⁺ TILs and TDLN of indicated treatment groups, replicate numbers as in f. h, Representative plot of memory CD8⁺ TILs and TDLN of indicated treatment groups (repeated×2). For a-c, treatment started when tumor volumes reached 400 mm³. For d-h, treatment started when tumor volumes reached 200 mm³. Average tumor volume (±s.e.m.) for each experimental group. Wilcoxon test was used for survival analysis. Flow cytometry as in materials and methods. Unpaired t-tests were performed, p-values 0.05 (*),0.01 (**), 0.001 (***) and 0.0001(****); p-values0.05 (ns).

### DETAILED DESCRIPTION OF THE INVENTION

In accordance with the present invention, a method is provided for treatment of prostate cancer that comprises administration to a patient in need of treatment a therapeutically effective amount of an IL-8 blocker in combination with androgen ablation. While the androgen ablation may result from bilateral orchiectomy, a generally clinically preferred method of androgen ablation is androgen deprivation therapy.

The androgen deprivation therapy may comprise administration of a drug to lower androgen levels such as an LHRH agonist (e.g., leuprolide, goserelin, triptorelin, or histrelin) or an LHRH antagonist (e.g., degarelix, a CYP17 inhibitor, or abiraterone); or a drug to stop androgen function, such as an anti-androgen (e.g., flutamide, bicalutamide, nilutamide, enzalutamide, or apalutamide). Other androgen suppressing drugs, such as an estrogen or ketoconazole may also be used. Although various exemplary androgen deprivation therapies are listed, it should be understood that the androgen deprivation therapy to be applied in the present invention is not limited to those listed herein and includes any form of androgen deprivation therapy presently known or to be developed in the future. The androgen deprivation therapy may be administered before, after, or simultaneously with the IL-8 blocker, but administration of ADT after administration of the IL-8 blocker is preferred. The androgen deprivation therapy may be used alone or in combination with bilateral orchiectomy. Those of skill in the oncological art will readily understand how to administer androgen deprivation therapy.

Thus, in one aspect of the invention, the disclosure provides a method of treating prostate cancer in a patient in need of such treatment which comprise administering to said patient in combination with androgen ablation a therapeutically effective amount of an IL-8 blocker, wherein the androgen ablation is selected from a bilateral orchiectomy and administration of an effective androgen ablation amount of a compound selected from an LHRH agonist (leuprolide, goserelin, triptorelin, or histrelin), an LHRH antagonist (degarelix, a CYP17 inhibitor, or abiraterone); an anti-androgen (flutamide, bicalutamide, nilutamide, enzalutamide, or apalutamide); an estrogen; ketoconazole; or a combination thereof.

The IL-8 blocker may be BMS-986253 (HuMax IL-8), ABX-IL8, HuMab 10F8, SCH527123/MK-7123, AZD5069, AZD5122, AZD8304, RIST4721, CCX832, a CX3CR1 antagonist, a CXCR1/2 monoclonal IL-8 blocker, a CXCR2 antagonist, CXCR2 biparatopic nanobodies, a CXCR2 monoclonal IL-8 blocker, DF1970, DF2726A, DF2156A, DF2162, DF2755A, repertaxin, reparixin, FX68, GSK1325756, GSK1325756H, SB225002, SB251353, SB332235, SB656933, KB03, MGTA145, PACG31P, PS291822 (navarixin), SX576, SX682, or other human ELR+CXC chemokine blockers. However, this list of possible IL-8 blockers is not considered to be limiting and other IL-8 (CXCL8) antagonists or IL-8 receptor (CXCR1/CXCR2) antagonists known or discovered in the future may be used in the present method. See, for example, Cheng, et al.—Potential Roles and Targeted Therapy of the CXCLs/CXCR2 axis in Cancer and Inflammatory Diseases, BBA-Reviews on Cancer 1871 (2019) pp 289-312, which is incorporated herein in its entirety.

Thus, one aspect of the invention provides a method of treating prostate cancer in a patient in need of such treatment which comprises administering to said patient in combination with androgen ablation a therapeutically effective amount of a compound selected from an IL-8 antagonist and an IL-8 receptor antagonist (collectively an “IL-8 blocker”), wherein the IL-8 blocker is selected from BMS-986253 (HuMax IL-8), ABX-IL8, HuMab 10F8, SCH527123/MK-7123, AZD5069, AZD5122, AZD8304, RIST4721, CCX832, a CX3CR1 antagonist, a CXCR1/2 monoclonal IL-8 blocker, a CXCR2 antagonist, CXCR2 biparatopic nanobodies, a CXCR2 monoclonal IL-8 blocker, DF1970, DF2726A, DF2156A, DF2162, DF2755A, repertaxin, reparixin, FX68, GSK1325756, GSK1325756H, SB225002, SB251353, SB332235, SB656933, KB03, MGTA145, PACG31P, PS291822 (navarixin), SX576, SX682, and other human ELR+CXC chemokine blockers.

Another aspect of the invention provides a method of treating prostate cancer in a patient in need of such treatment which comprises administering to said patient in combination with androgen ablation an amount of a compound selected from an IL-8 antagonist and an IL-8 receptor antagonist (an IL-8 blocker), the amount of the IL-8 blocker being effective to at least reduce the level of infiltration of suppressive immune cells into prostate tumor.

The androgen ablation may be selected from bilateral orchiectomy and administration of a drug to lower androgen levels such as an LHRH agonist (e.g., leuprolide, goserelin, triptorelin, or histrelin) or an LHRH antagonist (e.g., degarelix, a CYP17 inhibitor, or abiraterone); or a drug to stop androgen function, such as an anti-androgen (e.g., flutamide, bicalutamide, nilutamide, enzalutamide, or apalutamide). Other androgen suppressing drugs, such as an estrogen or ketoconazole may also be used.

The IL-8 blocker may be selected from BMS-986253 (HuMax IL-8), ABX-IL8, HuMab 10F8, SCH527123/MK-7123, AZD5069, AZD5122, AZD8304, RIST4721, CCX832, a CX3CR1 antagonist, a CXCR1/2 monoclonal IL-8 blocker, a CXCR2 antagonist, CXCR2 biparatopic nanobodies, a CXCR2 monoclonal IL-8 blocker, DF1970, DF2726A, DF2156A, DF2162, DF2755A, repertaxin, reparixin, FX68, GSK1325756, GSK1325756H, SB225002, SB251353, SB332235, SB656933, KB03, MGTA145, PACG31P, PS291822 (navarixin), SX576, SX682, and human ELR+ CXC chemokine blockers.

Other therapies may be used in combination with the disclosed method, including radiotherapy, immunotherapy, and chemotherapy, but immunotherapy is preferred. Preferred immunotherapy agents are anti-PD-1, anti-PD-L1, anti-CTLA-4, anti TIM-3, anti-TGIT, anti-CD40, a TLR agonist, a STING agonist, bi-specific T cell engagers (BiTEs) and dual-affinity retargeting antibodies (DARTs), chimeric antigen receptors (CARs) T cells, and an anti-cancer vaccine. The anti-cancer vaccine may be, for example, Sipuleucel-T, PSA-TRICOM, AVAX Tech vaccines, Prostvac-VF, or a listeria-based vaccine such as live attenuated double-deleted (LADD) and ADXS031-142.

In another aspect of the invention, the disclosure provides a method of treating prostate cancer in a patient in need of such treatment which comprise administering to said patient a therapeutically effective amount of an IL-8 blocker in combination with androgen ablation and a therapeutically effective amount of an immunotherapeutic agent selected from anti-PD-1, anti-PD-L1, anti-CTLA-4, anti TIM-3, anti-TGIT, anti-CD40, a TLR agonist, a STING agonist, bi-specific T cell engagers (BiTEs) and dual-affinity retargeting antibodies (DARTs), chimeric antigen receptors (CARs) T cells, and an anti-cancer vaccine. The anti-cancer vaccine may be selected from, for example, Sipuleucel-T, PSA-TRICOM, AVAX Tech vaccines, Prostvac-VF, and a listeria-based vaccine selected from live attenuated double-deleted (LADD) and ADXS031-142. The IL-8 blocker and the immunotherapy agent are preferably administered to the patient before administration of the ADT, although other orders of administration are possible as may be determined by one of skill in the art.

### Definitions

It is to be understood that the claimed methods are not limited to the particular embodiments described, and as such may vary. It is also to be understood that the terminology used herein is for the purpose of describing particular embodiments only, and is not intended to be limiting. The scope of the present technology will be limited only by the appended claims.

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

As used herein, the term “comprising” is intended to mean that the compositions and methods include the recited elements, but do not exclude others. “Consisting essentially of” when used to define compositions and methods, shall mean excluding other elements of any essential significance to the composition or method. “Consisting of” shall mean excluding more than trace elements of other ingredients for claimed compositions and substantial method steps for the claimed methods. Embodiments defined by each of these transition terms are within the scope of this disclosure. Accordingly, it is intended that the methods and compositions may include additional steps and components (comprising) or alternatively may include steps and compositions of no significance (consisting essentially of) or alternatively, may include only the stated method steps or compositions (consisting of).

As used herein, “about” means plus or minus 10%.

As used herein, the term “concurrently” with regard to administration of two or more therapeutic materials or modalities means that the two or more therapies or modalities are performed at about the same time. The two or more therapies or modalities may be performed simultaneously or successively, as will be understood by one skilled in the art.

As used herein, the phrase “in combination with” regarding two or more therapies or modalities and a patient means that the two or more therapies or modalities are administered to or performed on the patient with the intention that they have overlapping periods of efficacy. The two or more therapies or modalities may be performed concurrently or one may be performed before or after one another.

As used herein, “optional” or “optionally” means that the subsequently described event or circumstance may or may not occur, and that the description includes instances where said event or circumstance occurs and instances where it does not.

As used herein, the terms “individual”, “patient”, or “subject” can be an individual organism, a vertebrate, a mammal (e.g., a bovine, a canine, a feline, or an equine), or a human. In a preferred embodiment, the individual, patient, or subject is a human.

As used herein, the phrases “therapeutically effective amount” and “therapeutic level” mean a dose or plasma concentration of a therapeutic material in a subject or patient that provides the specific pharmacological effect for which the material is administered to the subject or patient in need of such treatment, i.e., to reduce, ameliorate, or eliminate prostate cancer. It is emphasized that a therapeutically effective amount or therapeutic level of a drug will not always be effective in treating prostate cancer, even though such dosage is deemed to be a therapeutically effective amount by those of skill in the art. The therapeutically effective amount may vary based on the route of administration and dosage form, the age and weight of the subject, and/or the subject's condition, including the stage of the cancer at the time that treatment commences, among other factors.

The terms “treatment” or “treating” as used herein with reference to prostate cancer, refer to reducing, ameliorating or eliminating one or more symptoms or effects of the disease. Response indicators that indicate the effects of treatment include a decline in prostate specific antigen (PSA) levels, tumor shrinkage, results in a bone-scan-based assay, pathologic complete response, surgical margin rates, and the like, as described in (e.g.) Teo et al.—“Drug development for noncastrate prostate cancer in a changed landscape” Nature Reviews (Clinical Oncology) (March 2018) 15, 168-182, which is incorporated herein by reference in its entirety.

A “therapeutic response” mean an improvement in at least one measure of prostate cancer, such as those describe above.

The phrase “infiltration of suppressive immune cells into prostate tumor” means the presence of myeloid-derived suppressor cells at the tumor microenvironment that may be positive for either CD33, CD15, CD66, or CD10 as determined by protein or RNA quantification using, but not limited to, at least one of the following methodologies: IF, IHC, flow cytometry, ELISA, CyTOF, CITE-Seq, PCR, RISH, RNAseq, or nanostring.

### Abbreviations

Dulbecco's Modified Eagles Medium (DMEM), Roswell Park Memorial Institute medium (RPMI); Fetal Bovine Serum (FBS); Charcoal Stripped Serum (CSS); Ribonucleic Acid (RNA); messenger RNA (mRNA); Deoxyribonucleic Acid (DNA); copy DNA (cDNA); Polymerase Chain Reaction (PCR); minute (min); second (sec); Androgen Receptor (AR); Androgen Deprivation Therapy (ADT); Institutional Animal Care and Use Committee (IACUC); Homeobox B13 (HoxB13); Green Fluorescent Protein (GFP); Testosterone (T); C-X-C Motif Chemokine Receptor 1 (CXCR1); C-X-C Motif Chemokine Receptor 2 (CXCR2); Cytotoxic T-lymphocyte Associated Protein 4 (CTLA4); Colony Stimulating Factor 1 Receptor (CSF1R); Intraperitoneal (IP); Subcutaneous (SQ); Tumor-Draining Lymph Nodes (TDLN); Tumor Infiltrating Lymphocytes (TILs); Tumor Associated Macrophages (TAMs); Polymorphonuclear (PMN); Myeloid-Derived Suppressor Cells (MDSCs); Low Density (LD); Red Blood Cells (RBCs); bi-specific T cell engagers (BiTEs); dual-affinity retargeting antibodies (DARTs), chimeric antigen receptors (CARs); listeria-based vaccines such as live attenuated double-deleted (LADD); Enzyme-Linked Immunosorbent Assay (ELISA); Fluorescenceactivated Cell Sorting (FACS); Immunohistochemistry (IHC); RNA In Situ Hybridization (RISH); Horseradish Peroxidase (HRP); Mouse Transcription Array (MTA); Robust Multi-Array Average (RMA); Gene Set Enrichment Analysis (GSEA); Molecular Signature Database (MSigDB); Integrative Genomics Viewer (IGV); CellTrace Violet (CTV); Immunohistochemistry (IHC); Mass Cytometry (CyTOF); and Cellular Indexing of Transcriptomes and Epitopes by Sequencing (CITE-Seq). “Nanostring” refers to technologies for protein and RNA quantification sold by Nanostring Technologies, Inc. Seattle, WA.

Amino acids are represented by the IUPAC abbreviations, as follows: Alanine (Ala), Arginine (Arg), Asparagine (Asn), Aspartic acid (Asp), Cysteine (Cys), Glutamine (Gin), Glutamic acid (Glu), Glycine (Gly), Histidine (His), Isoleucine (Ile), Leucine (Leu), Lysine (Lys), Methionine (Met), Phenylalanine (Phe), Proline (Pro), Serine (Ser), Threonine (Thr), Tryptophan (Trp), Tyrosine (Tyr), Valine (Val). Similarly for nucleotides: Adenine (A), Cytosine (C), Guanine (G), Thymine (T), Uracil (U), Adenine or Guanine (R), Cytosine or Thymine (Y), Guanine or Cytosine (S), Adenine or Thymine (W), Guanine or Thymine (K), Adenine or Cytosine (M), Cytosine or Guanine or Thymine (B), Adenine or Guanine or Thymine (D), Adenine or Cytosine or Thymine (H), Adenine or Cytosine or Guanine (V), and any base (N).

### Pharmaceutical Formulations

Pharmaceutical compositions suitable for use in the methods described herein may include an IL-8 blocker and a pharmaceutically acceptable carrier or diluent.

The composition may be formulated for intravenous, subcutaneous, intraperitoneal, intramuscular, oral, nasal, pulmonary, ocular, or rectal administration, but parenteral administration (such as intravenous) is preferred. In some embodiments, the IL-8 blocker are formulated for intravenous, subcutaneous, intraperitoneal, or intramuscular administration, such as in a solution, suspension, emulsion, liposome formulation, etc. The pharmaceutical composition can be formulated to be an immediate-release composition, sustained-release composition, delayed-release composition, etc., using techniques known in the art.

Pharmacologically acceptable carriers for various dosage forms are known in the art. For example, excipients, lubricants, binders, and disintegrants for solid preparations are known; solvents, solubilizing agents, suspending agents, isotonicity agents, buffers, and soothing agents for liquid preparations are known. In some embodiments, the pharmaceutical compositions include one or more additional components, such as one or more preservatives, antioxidants, stabilizing agents and the like. Pharmaceutically-acceptable carriers are well-known in the art and a suitable one can be selected by one of skill in the medical field. See, for example, Remington—The Science and Practice of Pharmacy (22^{nd} ed., 2012), Lloyd Allen, Jr., ed, which is incorporated herein by reference in its entirety.

Additionally, the disclosed pharmaceutical compositions can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In some embodiment, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound(s) in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

ADT and IL-8 blockers can be administered in combination with other therapeutics that are part of the current standard of care for prostate cancer. Alternatively, ADT and IL-8 blockers may be administered to a patient that has previously received conventional treatment for prostate cancer but who has not responded to conventional treatment (i.e., the disease is refractory or continues to progress).

### Methods of Treatment

In one aspect of the invention, disclosed herein is a method of treating prostate cancer in a patient in need of such treatment which comprises administering to said patient in combination with androgen ablation an amount of a compound selected from an IL-8 antagonist and an IL-8 receptor antagonist, the amount of said compound being effective to at least reduce the level of infiltration of suppressive immune cells into prostate tumor. The level of infiltration may be determined in pre- and post- treatment biopsy specimens by protein or RNA quantification using, but not limited to, at least one of the following methodologies: IF, IHC, flow cytometry, ELISA, CyTOF, CITE-Seq, PCR, RISH, RNAseq, or nanostring. Additionally, the presence (or absence) of MDSC in the primary tumor or in a biopsy of a metastatic lesion would also be a predictive biomarker of immune suppressive cell infiltration into the tumor.

In the present invention, at least one of an IL-8 antagonist or IL-8 receptor antagonist (“IL-8 blocker”) is administered in combination with androgen ablation (preferably ADT) to a patient (e.g., a human patient) suffering from prostate cancer to suppress or retard the effect of IL-8 in recruiting suppressor cells. The term “in combination” include administration before, concurrently with, or after the ADT. In some embodiments, the therapeutically effective amount of the IL-8 blocker is administered together with a pharmaceutically acceptable carrier. Suitable pharmaceutically acceptable carriers are well-known in the art. A typical route of administration is parenterally (e.g., intravenously, subcutaneously, or intramuscularly), as is well understood by those skilled in the medical arts. Other routes of administration are, of course, possible. Administration may be by single or multiple doses. The amount of IL-8 blocker administered and the frequency of dosing may be optimized by the physician for the particular patient.

Signs and symptoms of effective prostate cancer treatment may include, but are not limited to: a decline in prostate specific antigen (PSA) levels, tumor shrinkage, results in a bone-scan-based assay, pathologic complete response, surgical margin rates, and the like.

If the IL-8 blocker is a small molecule (such as a tyrosine kinase inhibitor) it is preferably administered orally, but if the IL-8 blocker is an antibody, it is preferably administered parenterally (e.g., intravenously or by subcutaneous injection) In some embodiment, the therapeutically effective dose of the IL-8 blocker may be administered 1 to 3 times a day if administrated orally or every 1 to 4 weeks if administrated intravenously.

In some embodiments, the effective orally-administered amount of the IL-8 blocker may be up to about 1,200 mg per kg; however, in some situations the dose may be higher or lower. In some embodiments, an effective orally-administered amount may be between about 5 g and about 100 g per day, between about 10 and about 90 g per day, between about 20 and about 80 g per day or any dose in between. In some embodiments, the effective dose administered intravenously may be from about 1 mg/kg to about 50 mg/kg. Those of skill in the cancer treatment art would readily understand how to adjust the dosage of the IL-8 blocker to achieve the intended effect.

### Methods of Treatment

The disclosed methods of treatment many also be combined with other known methods of treatment as the situation may require. These other methods of treatment include immunotherapy, chemotherapy, and radiotherapy, as are well-known in the oncological art.

### Experimental

### 1) Androgen Deprivation Therapy (ADT) Increases IL-8 Transcription in Prostate Cancer

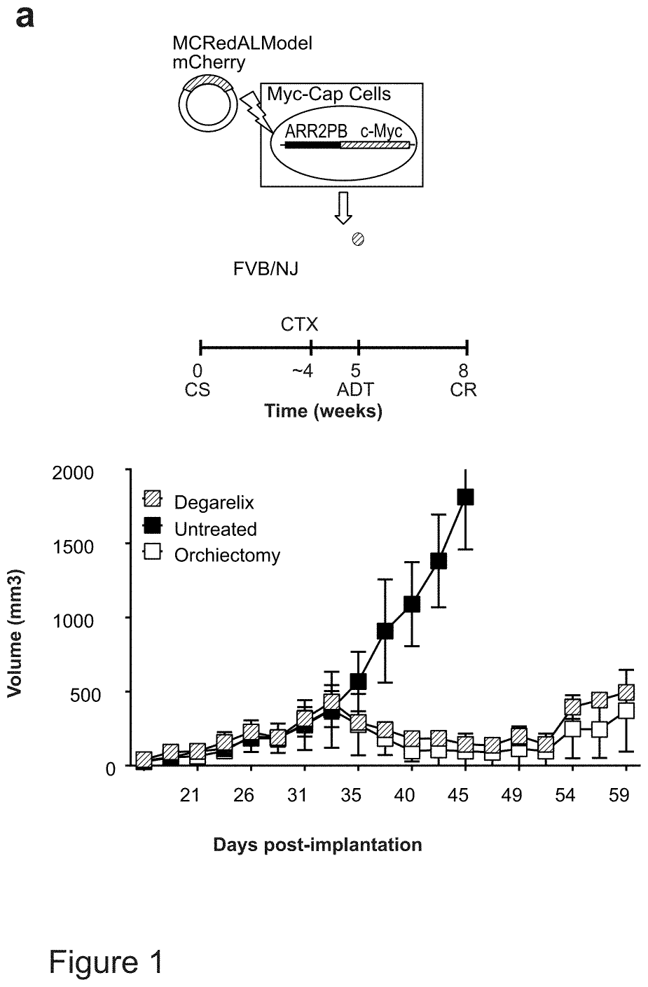

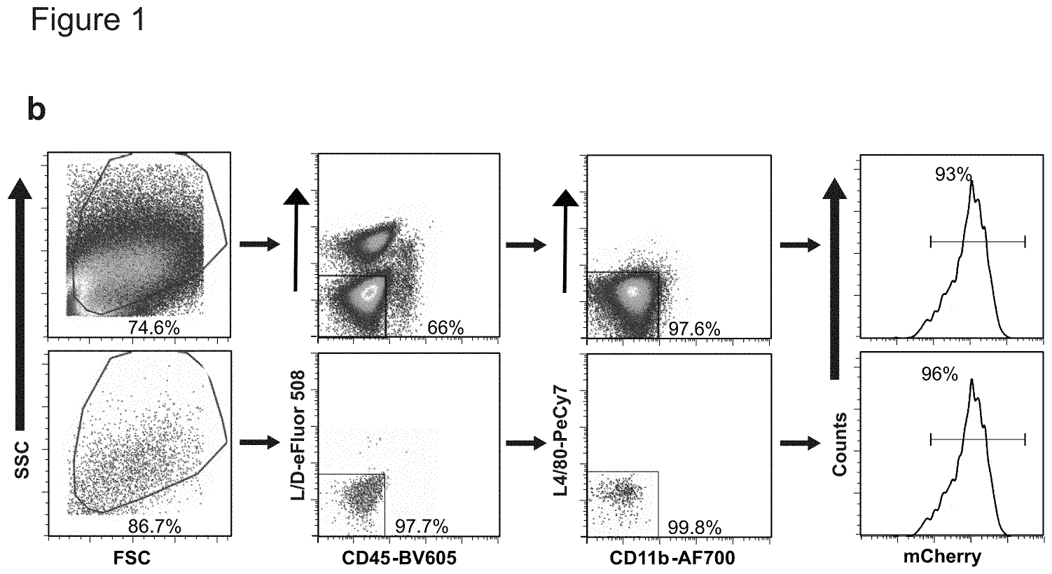

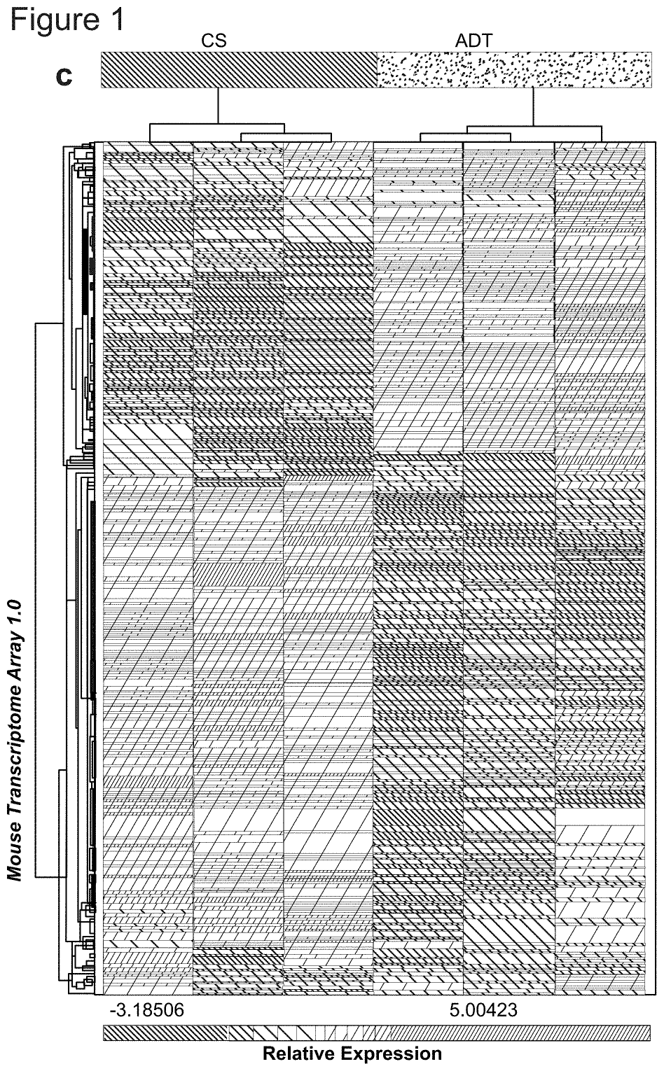

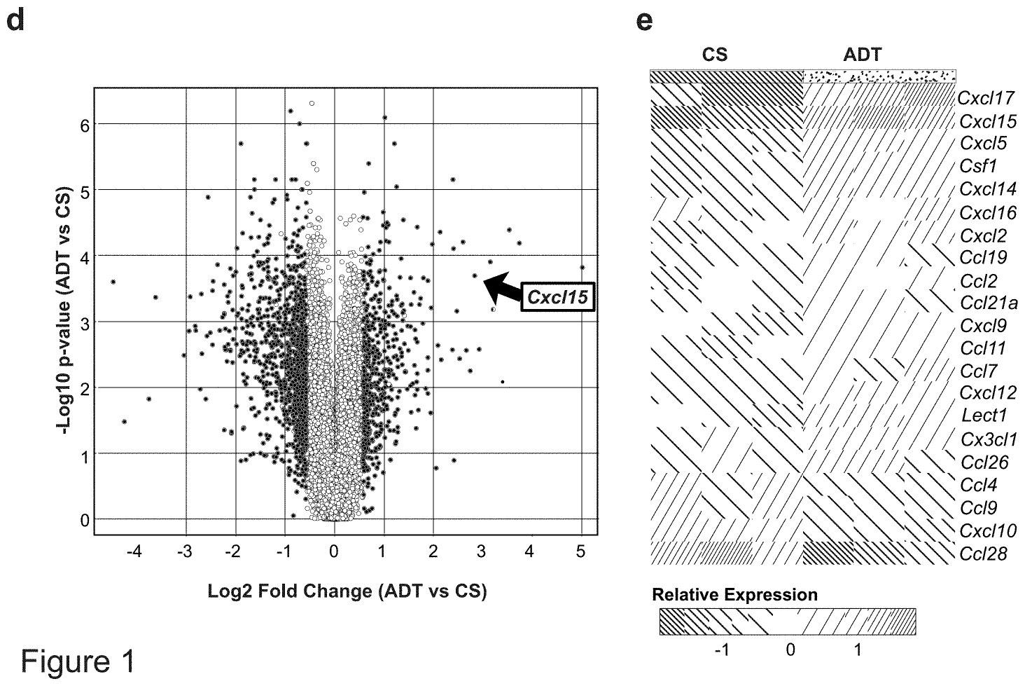

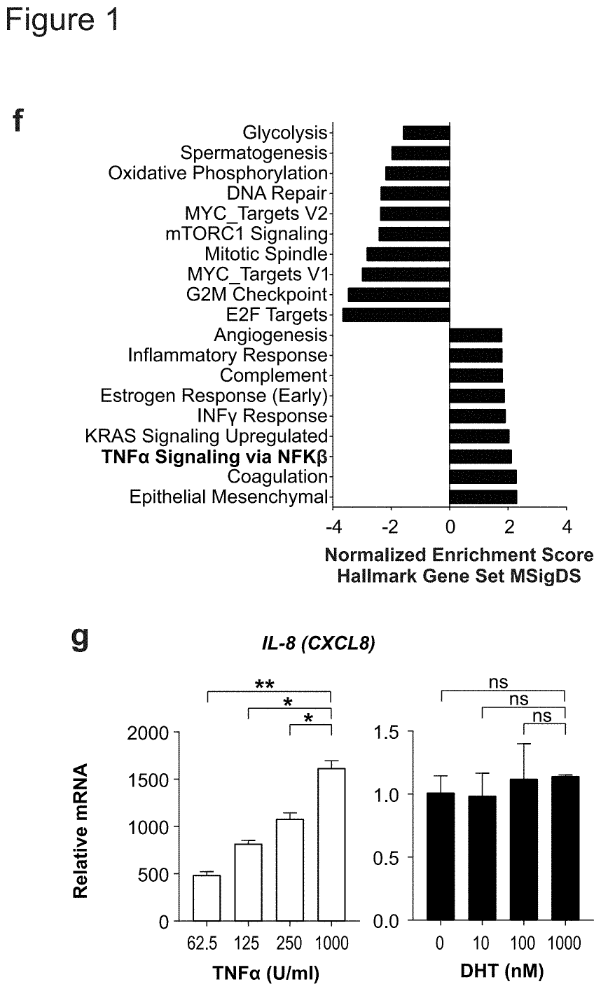

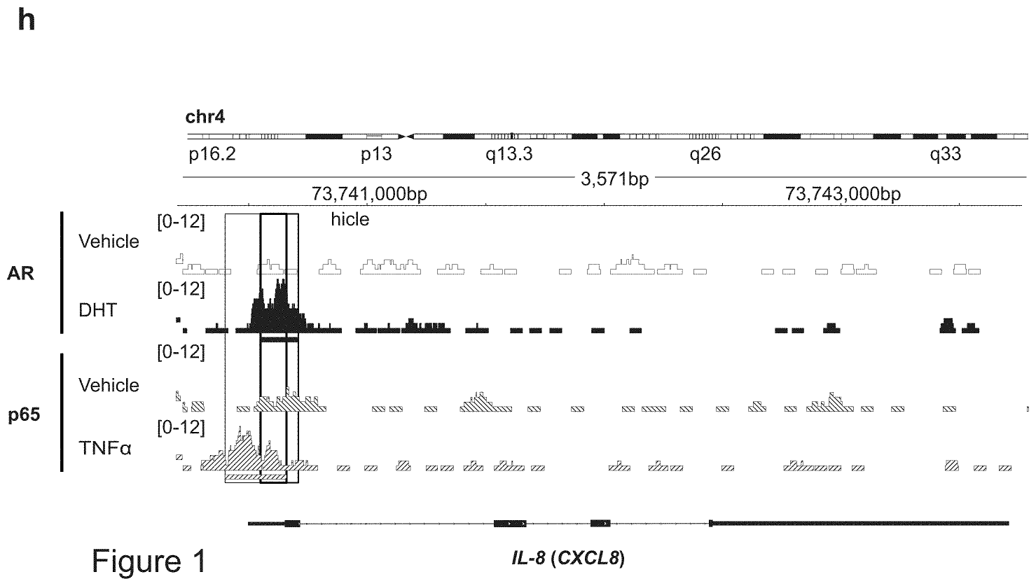

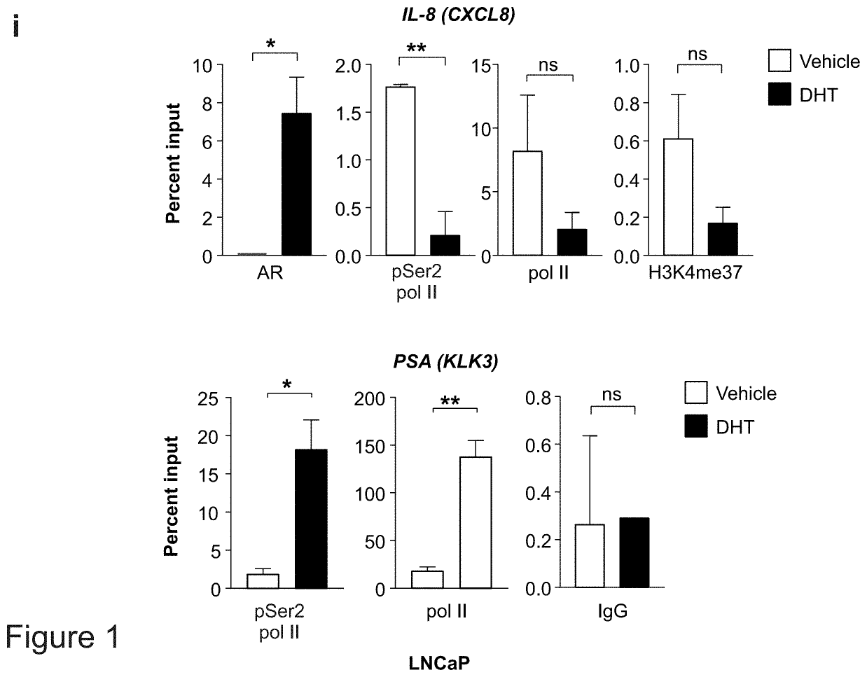

To identify immune-related tumor-cell intrinsic factors involved in prostate cancer progression, we performed expression analyses on murine prostate cancer cells pre- and post- castration. We used the MCRedAL prostate cancer cell line; an RFP expressing version of the Myc-Cap cell line characterized by MYC overexpression. Like human prostate cancer, MCRedAL tumors are initially castration-sensitive (CS), but castration-resistance (CR) develops approximately 30 days after castration (FIG. 1 a). Pre- and post- ADT tumor cells were sorted to >96% purity and analyzed (FIG. 1a-b). A number of cytokine and chemokine transcripts were significantly up-regulated post-ADT (FIG. 1d right), including Cxcl15, a CXC chemokine with a conserved ELR motif, which is the likely murine homolog of human IL-8 (CXCL8). qRT-PCR and ELISA assays confirmed the upregulation of Cxcl15 post-ADT at the protein level (data not shown). In addition to the chemokines above, GSEA revealed the upregulation of several pro-inflammatory pathways post-ADT (FIG. 1e). In vitro experiments using the human androgen-responsive LNCaP cell line corroborated a role for these pro-inflammatory signals, showing that in the absence of androgen, TNFα upregulated IL-8 expression in a dose-dependent manner (FIG. 1f left); while AR signaling in the absence of inflammation did not affect IL-8 expression (FIG. 1f right). These data led to the hypothesis that AR signaling directly suppresses IL-8 expression in prostate cancer cells. We performed in silico ChIP-Seq analyses using human LNCaP cells (GSE83860) and found AR binding at the IL-8 promoter in the presence of the potent androgen dihydrotestosterone (DHT; FIG. 1g top). This androgen dependent binding was verified by ChIP-qRT-PCR (FIG. 1h).

To further explore the role of AR in IL-8 regulation, we interrogated RNA polymerase binding and transcription marks found at sites of active promoters. In the presence of DHT, binding of RNA polymerase II (pol II), phosphorylated serine 2 RNA polymerase II (pSer2 pol II) and histone H3 tri-methyl Lys4 (H3K4me3) to the IL-8 locus were substantially reduced, consistent with reduced transcriptional activity (FIG. 1h left). Conversely, pSer2 pol II binding to the promoter of the well-established AR-regulated gene PSA (KLK3), was significantly increased in the presence of DHT as expected (FIG. 1h right). Consistent with a role for inflammation, TNFα significantly increased p65 binding at the IL-8 (CXCL8) promoter in LNCaP cells (FIG. 1g bottom). These data suggest that AR directly suppresses IL-8 expression through repressive AR binding to the IL-8 promoter. Taken together, we found that IL-8 transcription is up-regulated by pro-inflammatory signaling, and down-regulated by AR signaling.

### 2) IL-8 is Differentially Expressed in Castration Resistant Versus Castration Sensitive Prostate Cancer Cells.

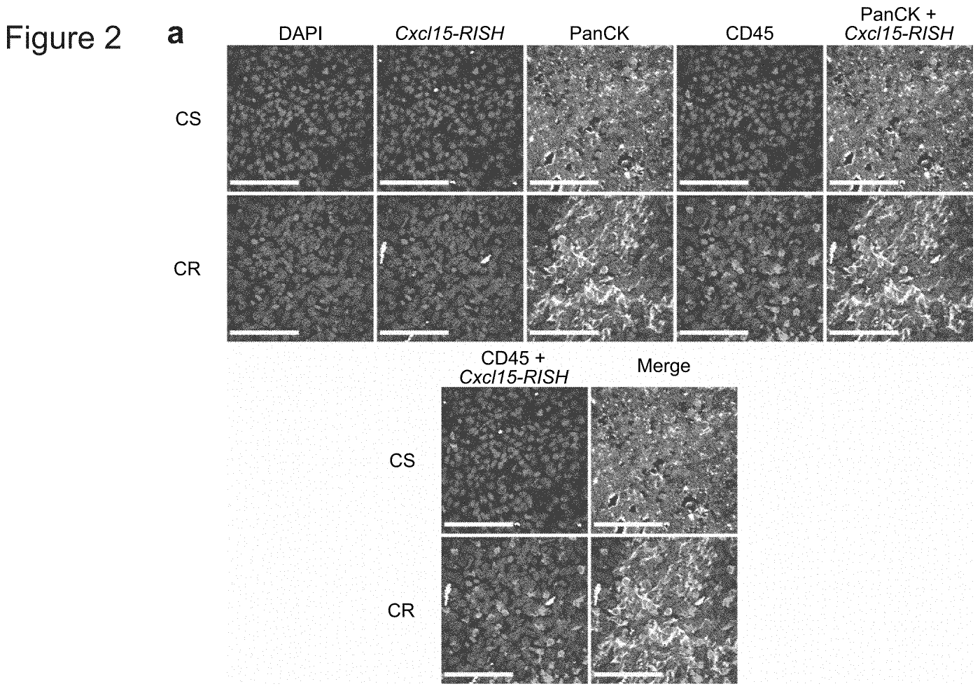

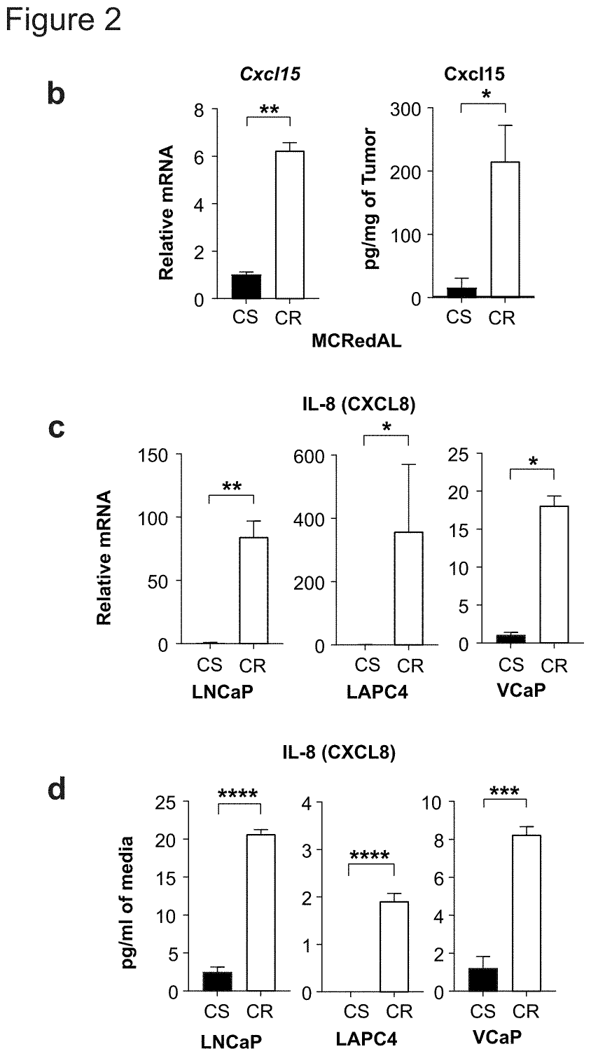

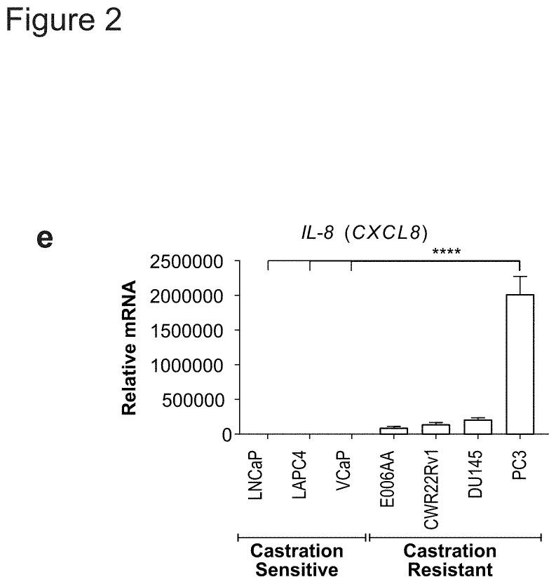

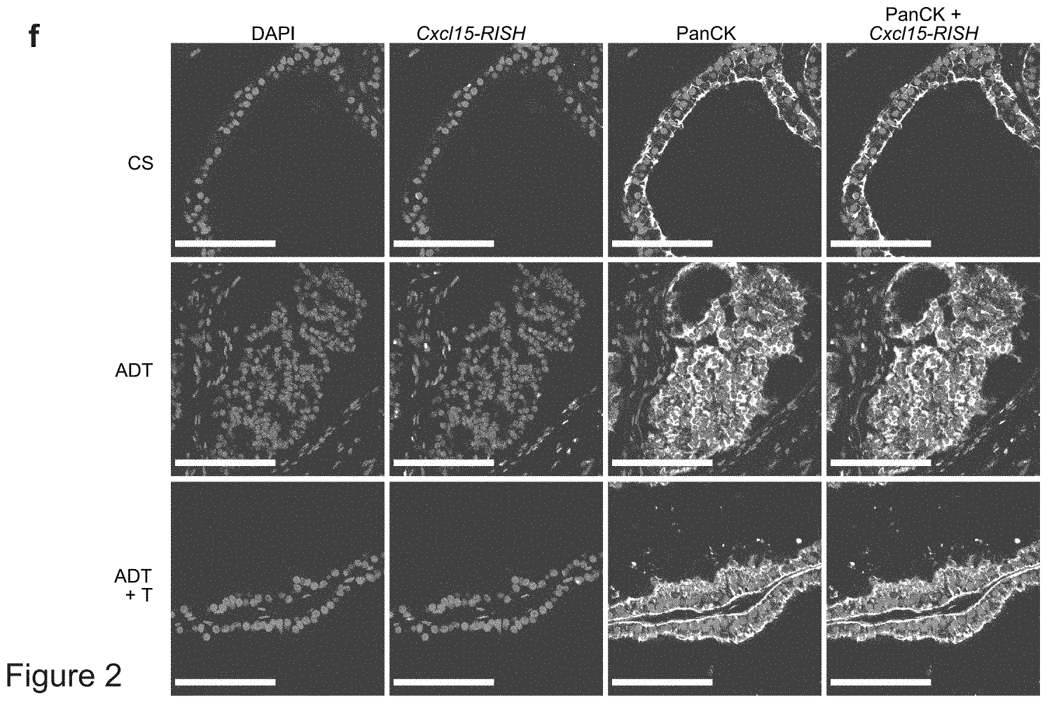

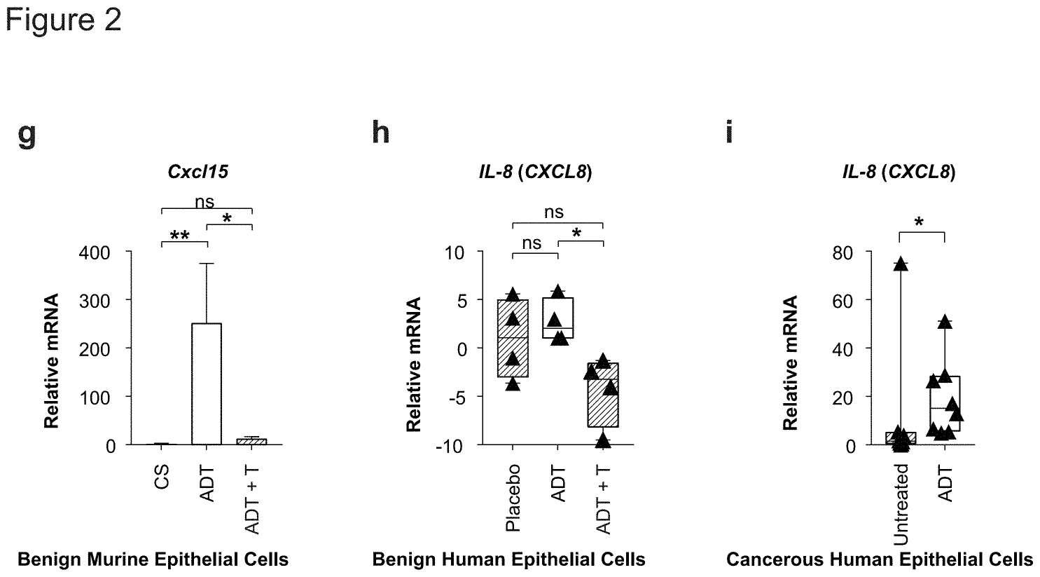

We next investigated the effects of ADT on the expression of Cxcl15 in vivo, using RNA in situ hybridization (RISH) to study Myc-Cap tumors. We found that CR tumors expressed increased Cxcl15 as compared to CS tumors, particularly in epithelial (PanCK⁺) tumor cells (FIG. 2a). These findings were confirmed in vitro, both at the mRNA and protein level (FIG. 2b). To investigate these findings in the context of human prostate cancer, we used three paired cell lines in which isogenic CR lines were derived from CS progenitors. For each pair, the CR line expressed significantly increased IL-8 as compared to the CS counterpart, both at the mRNA and protein level (FIG. 2c-d). This observation held across a panel of AR expressing prostate cancer cell lines; with higher levels of IL-8 expression in cell lines from castration-resistant disease (FIG. 2e). To test whether AR modulates Cxcl15 expression in benign prostate epithelium, we used RISH to study WT mice treated with ADT, and WT mice treated with ADT followed by testosterone (T) repletion. These data (FIG. 2f-g) showed increased epithelial Cxcl15 expression in ADT samples with expression significantly decreased by testosterone repletion (FIG. 2g). This observation was further corroborated by interrogating a dataset (GSE8466) profiling human prostate epithelial cells isolated by laser-capture microdissection (LCM) from men undergoing ADT and ADT with testosterone supplementation. Testosterone repletion significantly reduced IL-8 mRNA expression (FIG. 2h), supporting the hypothesis that AR signaling down-regulates IL-8 expression. In agreement with these data from benign prostate tissues, we LCM-enriched tumor prostate epithelium from high-risk PCa patients treated with ADT on a neo-adjuvant trial (NCT01696877) and found increased IL-8 expression as compared to tumors from age and stage-matched untreated controls (FIG. 2i). Taken together, analyses using human tissues strongly support the fact that castration increases IL-8 expression in prostate epithelial cells.

### 3) ADT-Driven IL-8 (and Cxcl15) Up-Regulation Promotes PMN-MDSC Infiltration.

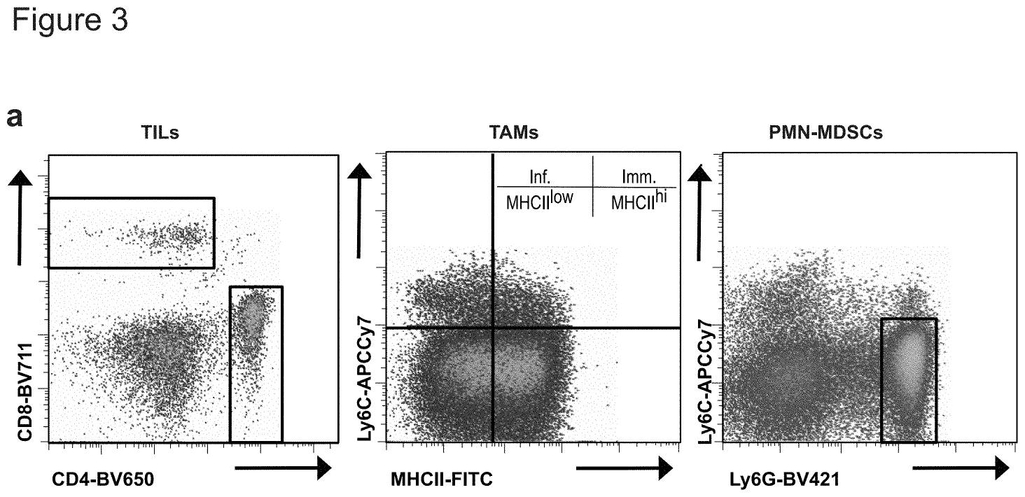

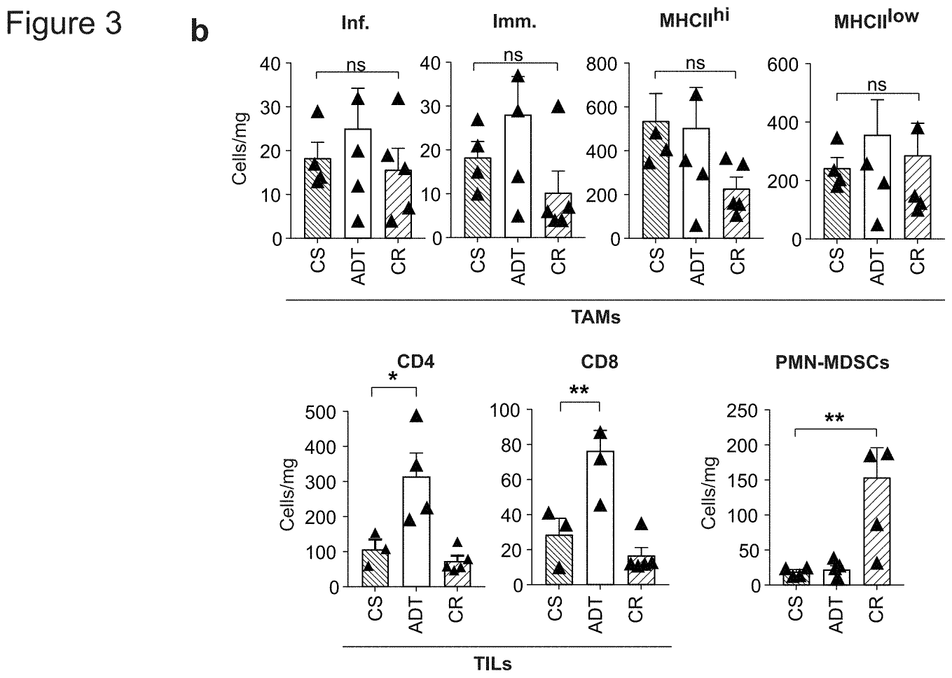

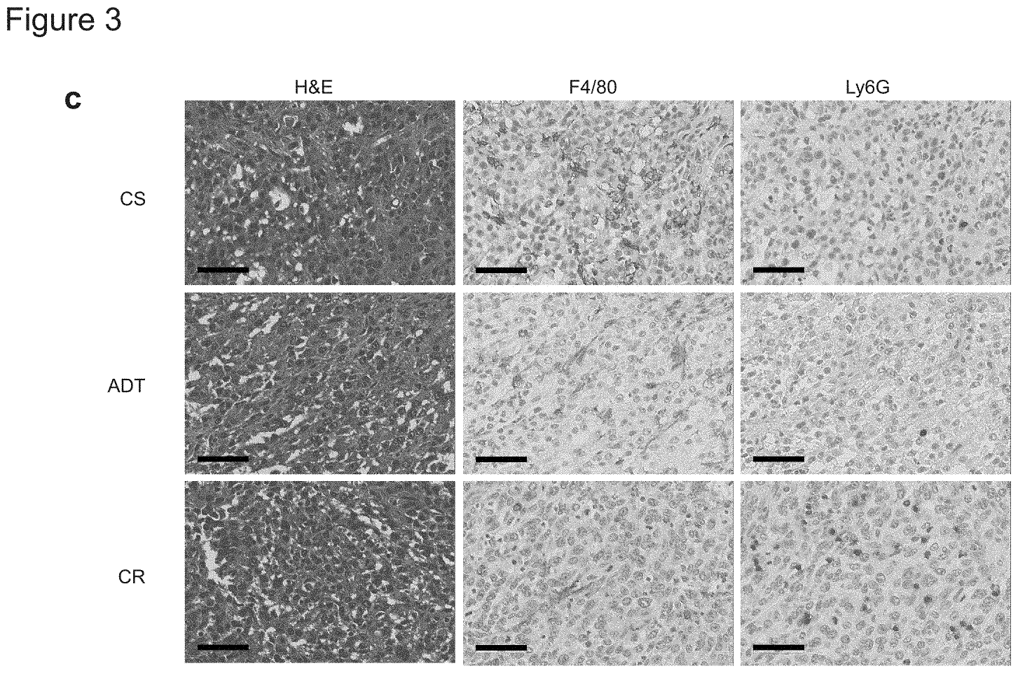

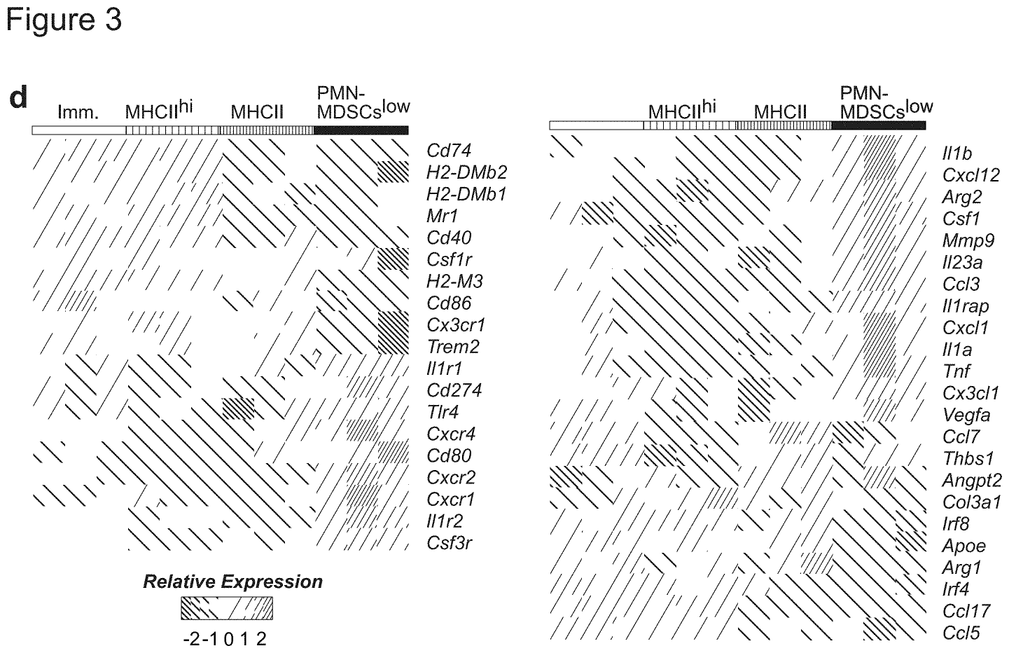

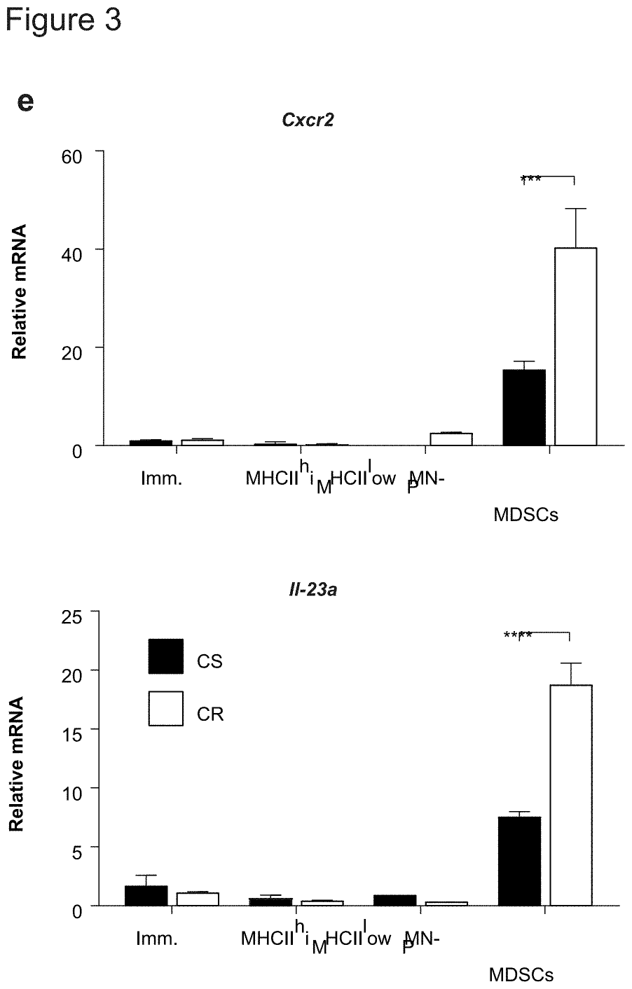

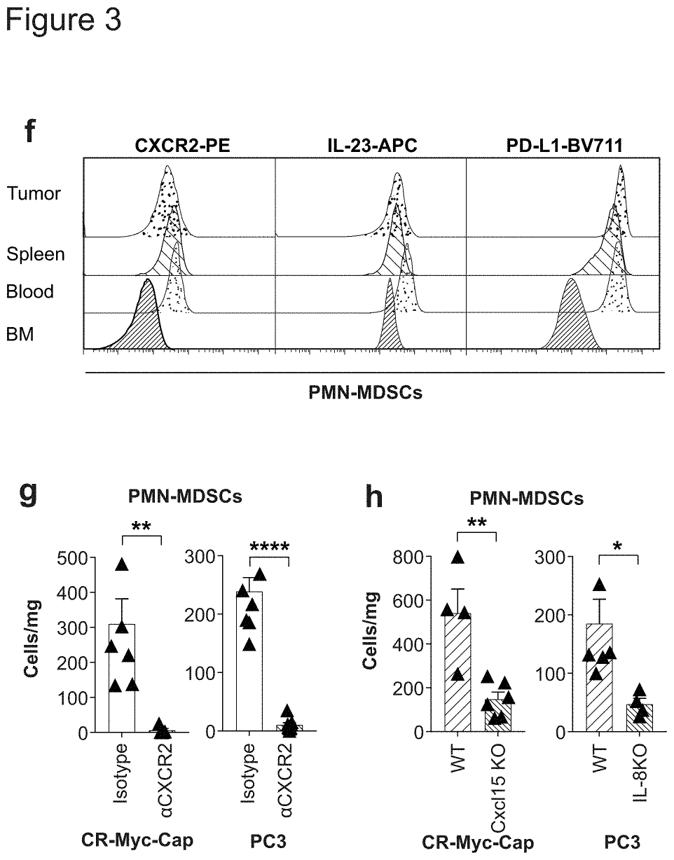

We next quantified castration-mediated immune infiltration in Myc-Cap allografts (FIG. 3a). Consistent with prior data, ADT promoted a transient T cell influx, without significant changes in tumor associated macrophage (TAM) populations (FIG. 3b). By contrast, PMN-MDSC infiltration was significantly increased in CR tumors (FIG. 3b), as verified by IHC (FIG. 3c). Molecular profiling of the infiltrating myeloid cells revealed a signature consistent with functional PMN-MDSCs, including up-regulation of IL-1b, Arg2 and IL-23a (FIG. 3d). In particular, increased expression of IL-23a and Cxcr2 was verified by qRT-PCR (FIG. 3e) and flow cytometry (FIG. 3f). To test whether blocking the IL-8/CXCR2 axis was sufficient to attenuate post-ADT PMN-MDSC infiltration, we treated prostate-tumor bearing mice with anti-CXCR2 and found that blocking CXCR2 significantly diminished tumor infiltration with PMN-MDSCs in both human (PC3) and murine (Myc-Cap) immunodeficient and immunocompetent models (FIG. 3g). To confirm this observation at the genetic level, we used CRISPR/Cas9 to generate human (PC3) and mouse (Myc-Cap) lines that were knocked out for human IL-8 or the murine IL-8 homolog Cxcl15, respectively. We observed a clear decrease in PMN-MDSC infiltration in both settings (FIG. 3h).

### 4) Blockade of The CXCR2/IL8 Pathway Attenuates the Migration of PMN-MDSCs But Not Their Function.

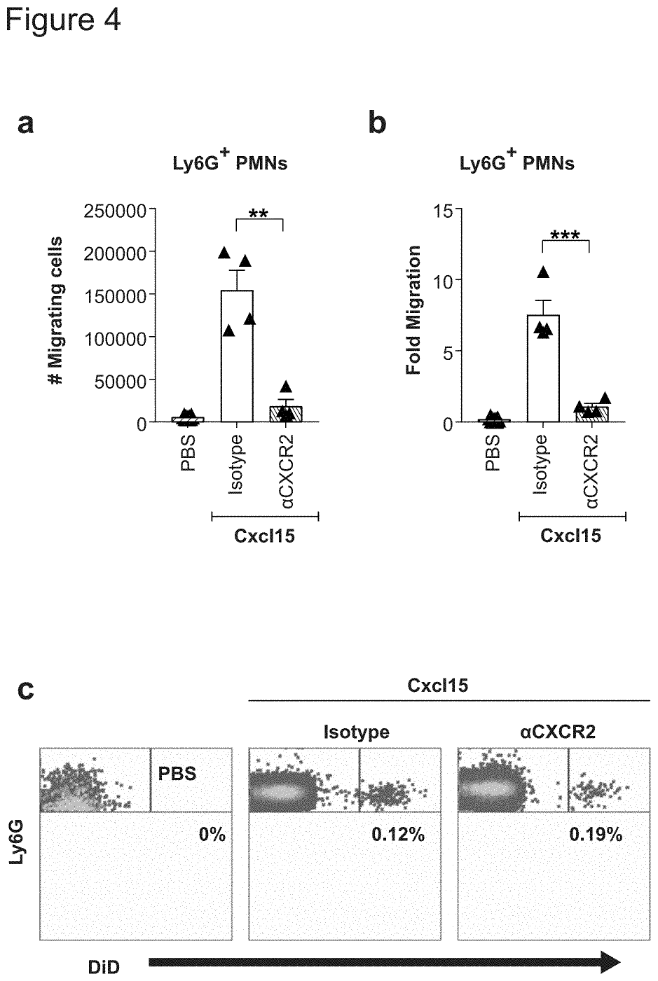

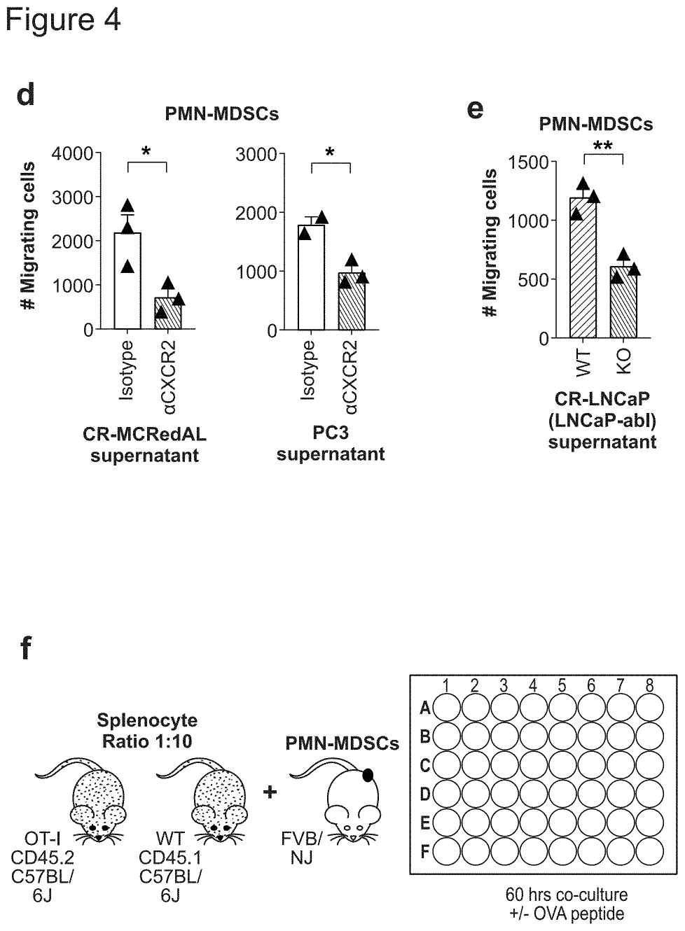

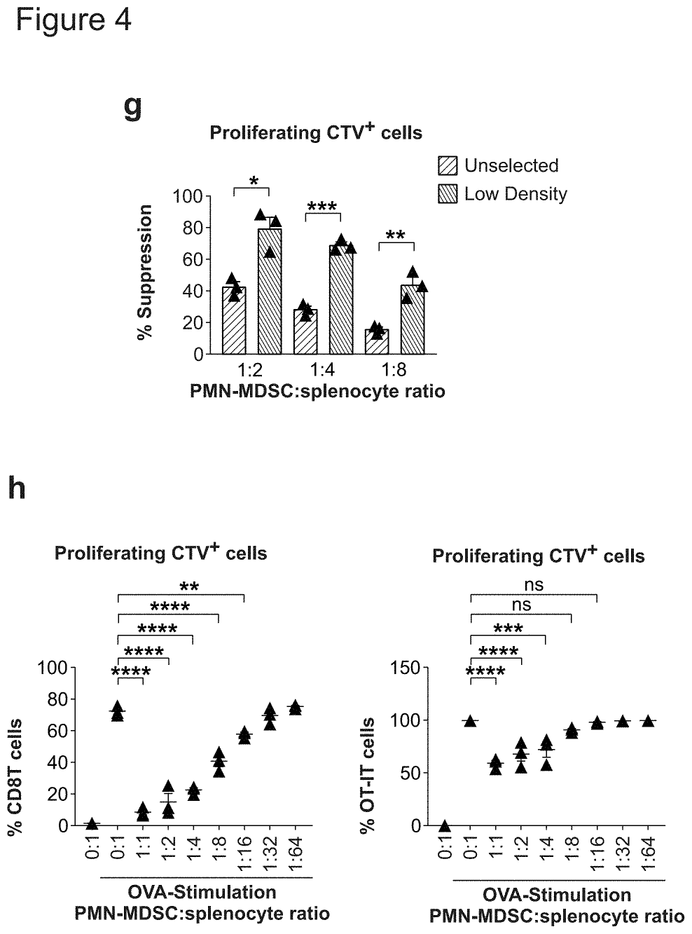

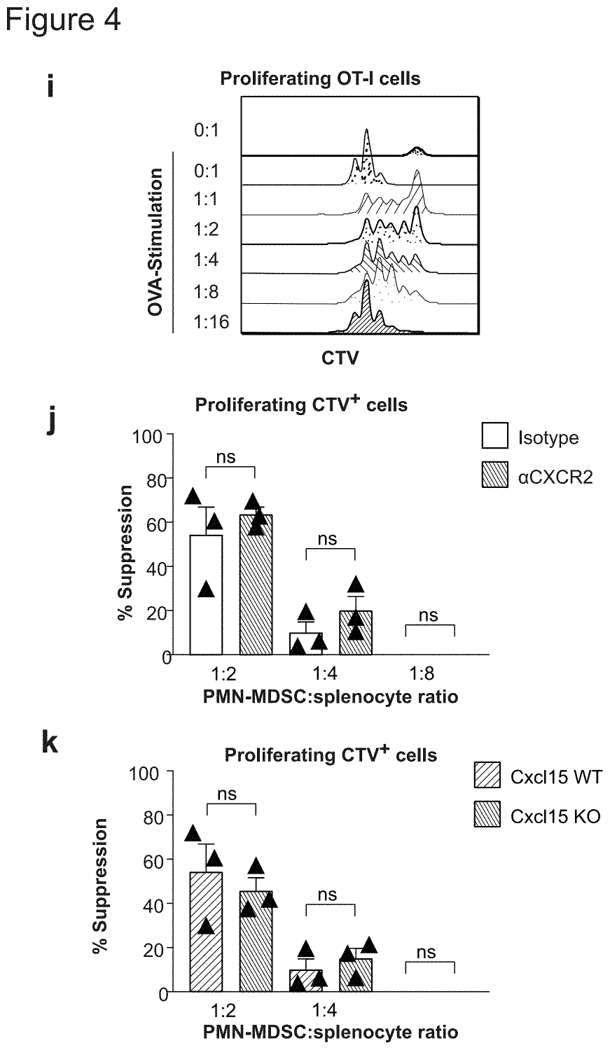

We next asked whether the supernatants from castration-resistant MCRedAL (CR-MCRedAL) cells were sufficient to drive PMN-MDSC migration in vitro. In line with in vivo results (FIG. 3g-h and FIG. 4a-c), we found that PMN-MDSC migrated towards the supernatant of CR tumors and migration was significantly attenuated by CXCR2 blockade (FIG. 4d). Human prostate cancer (PC3) showed an identical pattern. To confirm a role for IL-8 in PMN-MDSC migration, we generated IL-8 KO CR-LNCaP (LNCaP-abl) using CRISPR/Cas9. Supernatants from IL-8 KO cells were significantly attenuated in their ability to promote PMN-MDSC migration (FIG. 4e). These PMN-MDSCs were functional and suppressed CD8 T cell proliferation in a dose-dependent manner (FIG. 4f-i). Although CXCR2 blockade decreased PMN-MDSC migration, it did not significantly alter their suppressor function (FIG. 4j). Similarly, Cxcl15 loss did not diminish the suppressive function of PMN-MDSCs (FIG. 4k). Taken together these findings reinforce a functional role for castration-mediated IL-8 secretion in PMN-MDSC migration.

### 5) CXCR2 Blockade Improves Response to Checkpoint Blockade Following Androgen-Deprivation in a Physiologically Relevant Model of Prostate Cancer.

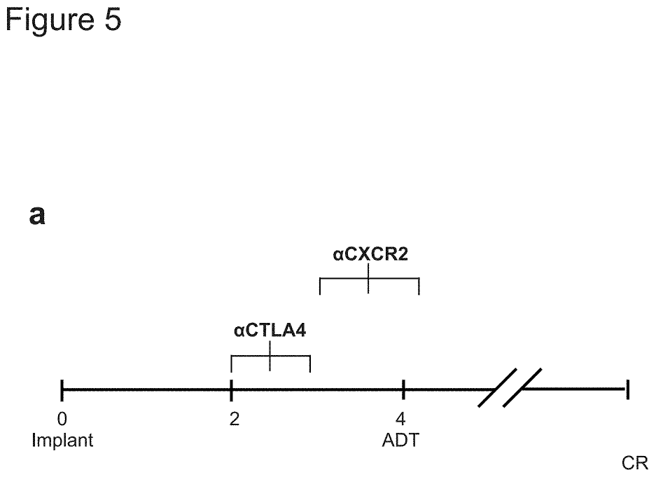

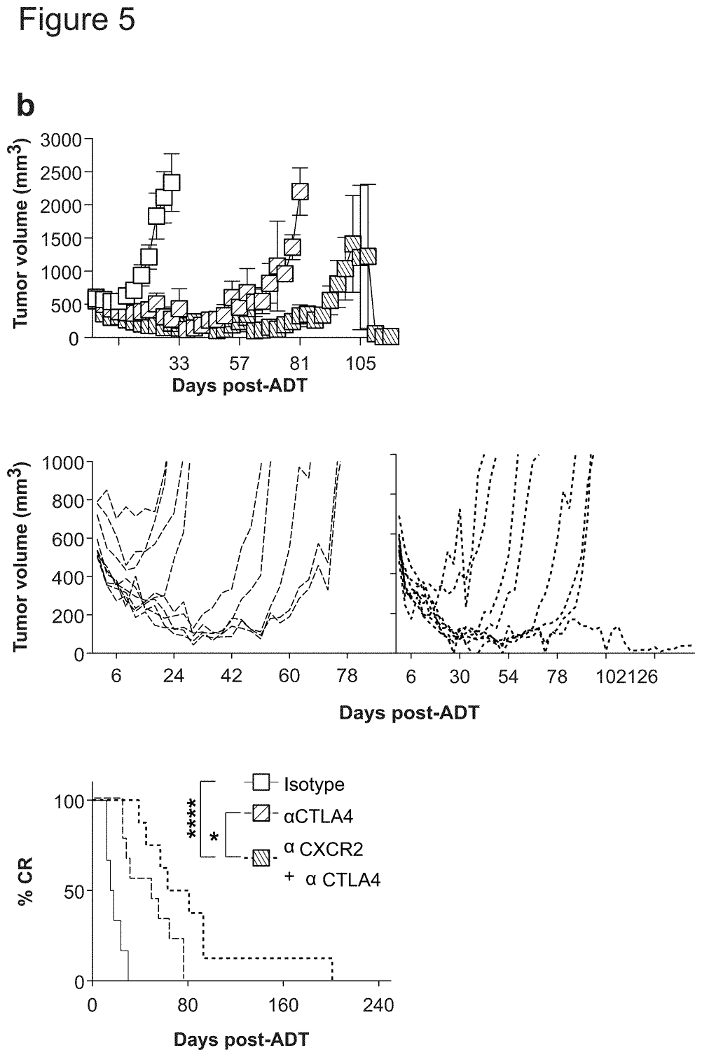

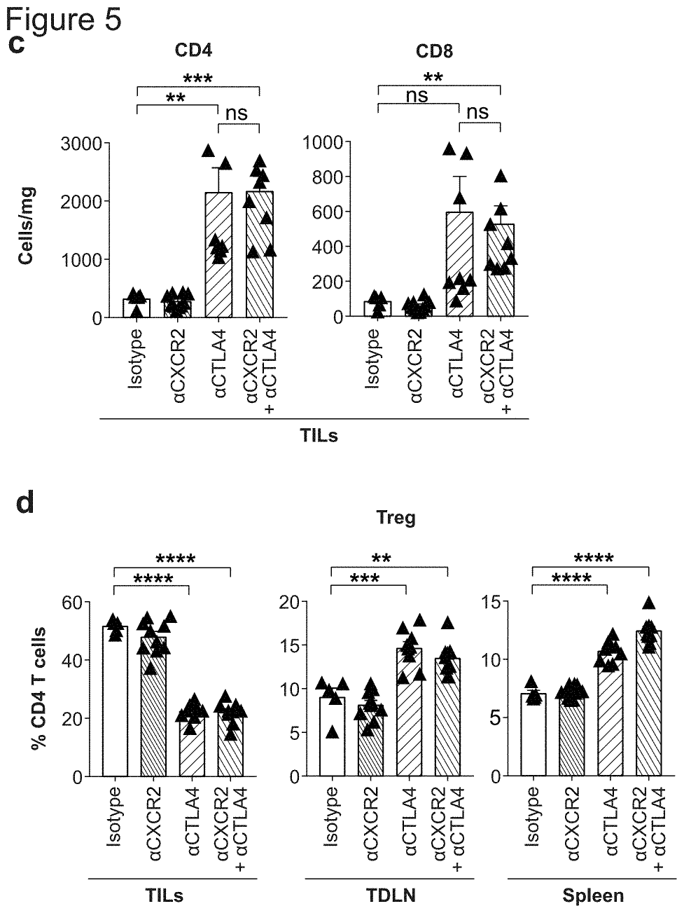

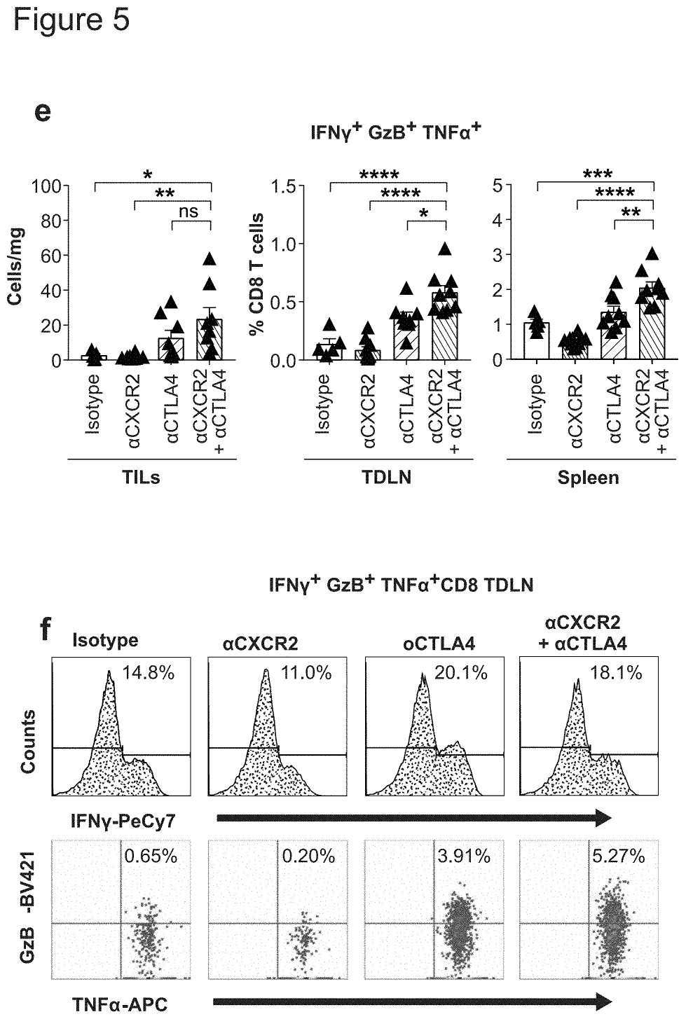

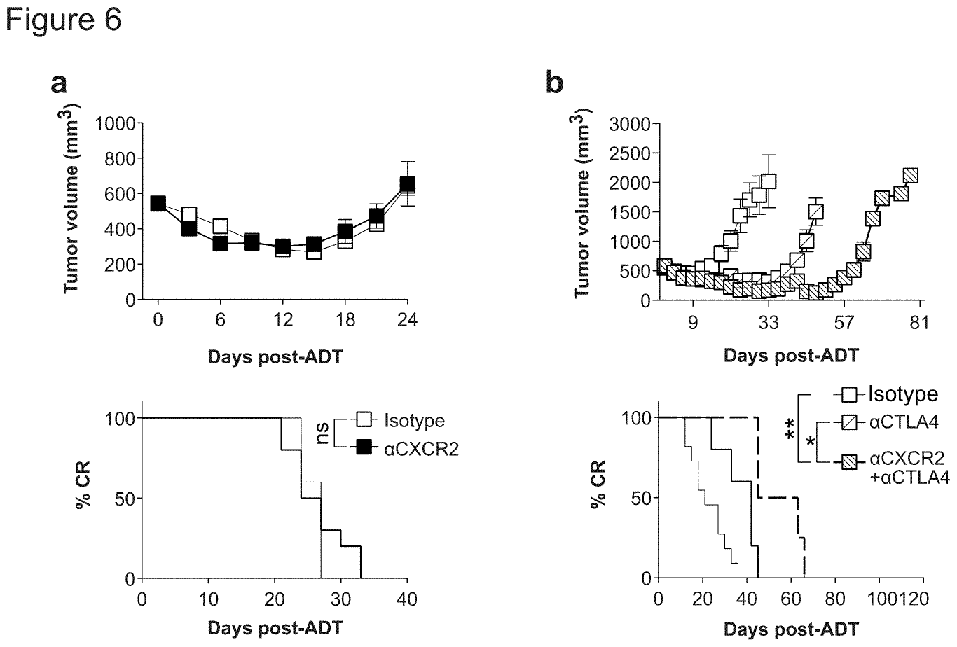

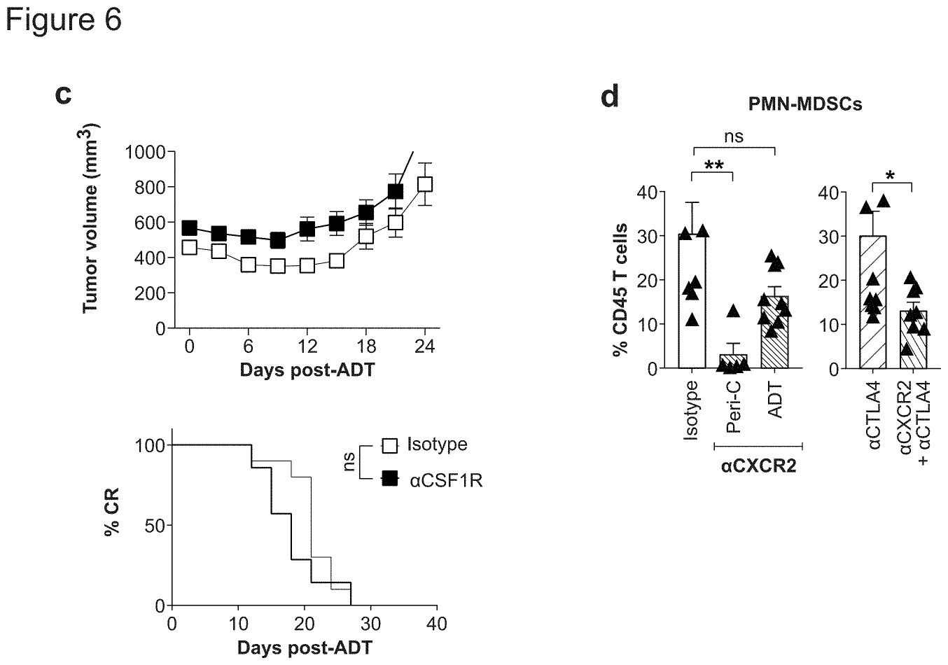

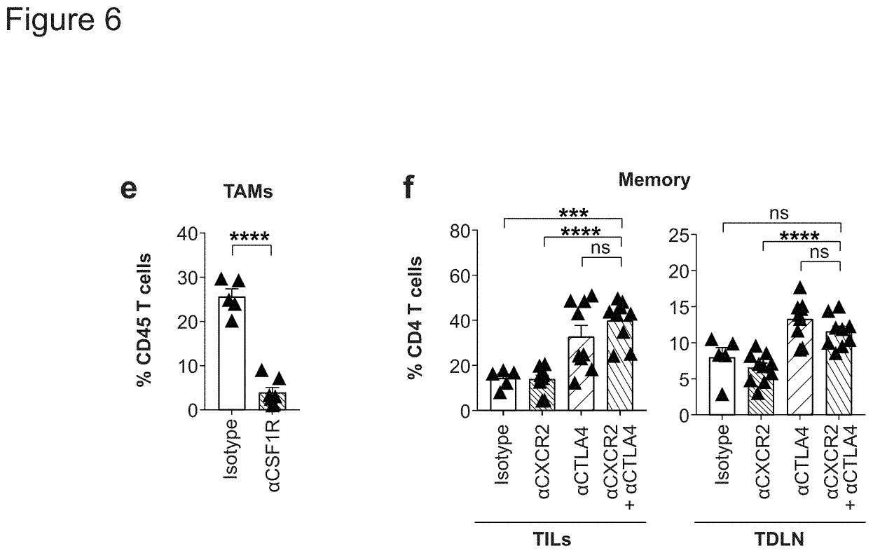

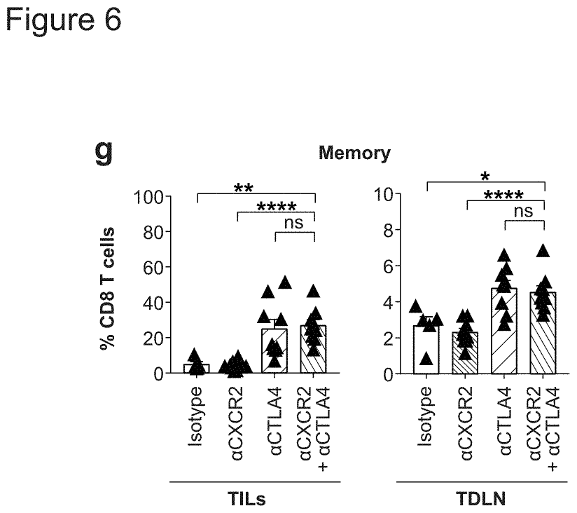

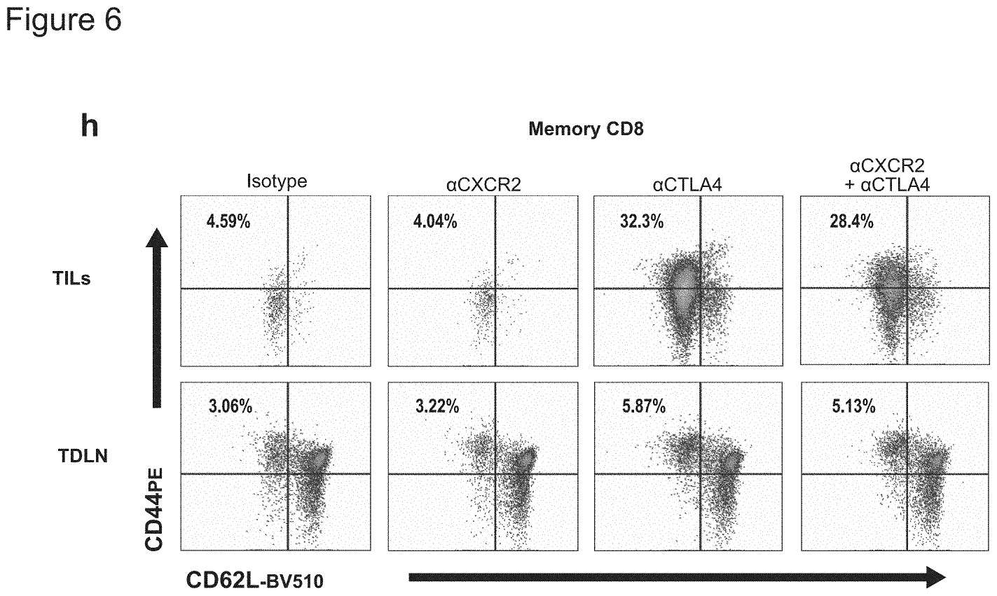

Finally, we investigated the pre-clinical activity of blocking the IL-8/CXCR2 axis at the time of androgen-deprivation in the Myc-Cap model. Notably, in the absence of immunotherapy the combination of ADT and CXCR2 blockade was less effective (FIG. 6a). In contrast, combining CXCR2 blockade with ICB (anti-CTLA-4; FIG. 5a) resulted in significantly increased survival (FIG. 5b). This triple combination (ADT+anti-CXCR2+anti-CTLA-4) was effective even when tumors were relatively advanced (400 mm³) at the time of treatment (FIGS. 6b&d). Macrophage modulation with anti-CSF1R was not effective therapeutically in this setting (FIGS. 6c&e). Mechanistically, the increased anti-tumor effects mediated by the addition of anti-CXCR2 to ADT +anti-CTLA-4 did not appear to be due to increased T cell infiltration (FIG. 5c and FIG. 6f-h), nor due to decreased Treg infiltration (FIG. 5d), but rather correlated with an increase in polyfunctional effector CD8 T cells in tumor-draining lymph nodes (TDLN) and spleens (FIGS. 5e&f).

The following examples are given to illustrate the present invention. It should be understood, however, that the invention is not to be limited to the specific conditions or details described in the example. All printed publications referenced herein are specifically incorporated by reference.

### EXAMPLES

### Patient Samples

Formalin fixed, paraffin embedded (FFPE) human prostate cancer samples were obtained from consented patients treated with ADT (degarelix; 240 mg SQ) in a neo-adjuvant trial (NCT01696877) and matched control radical prostatectomies were obtained from patients treated at the Johns Hopkins Sidney Kimmel Comprehensive Cancer Center (Baltimore, Md.) under IRB-approved clinical protocol J1265. All patients provided written, informed consent.

### Cell Lines

Myc-Cap, derived from spontaneous prostate cancer in c-Myc transgenic mice, was a generous gift from Dr. C. Sawyers. To generate MCRedAL, Myc-Cap cells were transfected with pRetroQ-mCherry-C1 (Clontech) using lipofectamine 2000 (Invitrogen) and isolated by FACS sorting based on mCherry expression (Extended Data FIG. 1a). Myc-Cap and MCRedAL cells were cultured in DMEM as previously described. LNCaP, VCaP, E006AA, CWR22Rv1, DU145, and PC3 cell lines were obtained and cultured as recommended by the ATCC. LAPC4 (a gift from Dr. S. Yegnasubramanian) were maintained in RPMI-1640 (Corning) supplemented with 10% fetal bovine serum (FBS; Gemini Bio-Products). Androgen independent LNCaP-abl cells were a gift from Dr. Z. Culig and cultured as described previously²⁹. LAPC4-CR and VCaP-CR (a gift from S. Yegnasubramanian) were derived by passaging LAPC4 and VCaP cells through castrated animals and further subculturing in RPMI-1640 supplemented with 10% charcoal stripped serum (CSS; Gemini Bio-Products) supplemented with 1×B-27 Neuronal Supplement (Gibco). For experiments when cells were grown in androgen-free conditions, 10% FBS was substituted for 10% CSS in complete media. For migration/chemotaxis assays, prostate cancer cell lines were cultured in complete media containing either 0.5% or 2.5% FBS for human and murine cells, respectively. All cell lines were cultured in 1% penicillin/streptomycin media at 37° C., 5% CO₂.

### Mouse Strains

Seven-week-old FVB/NJ, J:NU, C57BL/6-Tg(TcraTcrb)1100Mjb/J (OT-I), and B6.SJL-PtprcaPepcb/BoyJ (CD45.1) male mice were purchased from The Jackson Laboratory. A breeding pair of Hoxb13-rtTA\TetO-H2BGFP (HOXB13-GFP) mice was received from University of Maryland Baltimore County and experimental animals were bred in-house. Animals were kept in a specific pathogen-free facility at either Johns Hopkins University School of Medicine or Columbia University Medical Center. All animal experiments were performed in accordance with protocols approved by the Institutional Animal Care and Use Committee (IACUC) at the respective institutions.

### Tumor Allografts and Xenografts

Eight-week-old male FVB/NJ and J:NU mice were subcutaneously inoculated in the right flank with either Myc-Cap or MCRedAL (1×10⁶ cells/mouse), and LNCaP or PC3 (3×10⁶ cells/mouse), respectively. Tumor diameters were measured with electronic calipers every 3 days as indicated and the tumor volume was calculated using the formula: [longest diameter×(shortest diameter)²]/2. Myc-Cap tumor bearing mice received androgen-deprivation therapy (ADT) 4 weeks after tumor implantation when tumor volume reached ˜500 mm³, as indicated in figure legends. ADT was administered via subcutaneous (sc) injection of degarelix acetate (a GnRH receptor antagonist; Ferring Pharmaceuticals Inc.) at a dosage of 0.625 mg/100 μl H₂O/25 g body weight every 30 days, unless otherwise indicated. Onset of castration-resistance was defined as the time to tumor size increased by 30% (˜650 mm³) after ADT. Chemical castration by ADT was compared to bilateral orchiectomy as described in FIG. 1a.

### Luminal Epithelial Regression/Regeneration

Eight-week-old male HOXB13-GFP mice carrying the Hoxb13-rtTA transgene and a Tetracycline operator—Histone 2B-Green Fluorescent Protein (TetO-H2BGFP), which results on GFP expression being restricted to luminal epithelial Hoxb13⁺ cells (described previously), were castrated via bilateral orchiectomy. A cycle of prostate regression/regeneration was induced as described previously. Briefly, mice were allowed to regress for six weeks to reach the fully involuted state. Mice were randomized to ADT or ADT+testosterone (T) treatment groups. Testosterone was administered for four weeks for prostate regeneration by subcutaneous pellets; this regimen yields physiological levels of serum testosterone. All mice received 2 mg/ml of Doxycycline (Sigma) in the drinking water to induce GFP expression under the control of the luminal epithelial promoter, HoxB13, one week prior euthanizing them for their analysis.

### IL-8 Blocker Treatment

Anti-CXCR2 (murine IgG1-D265A, clone: 11C8; a non-FcγR-binding mutant with deficient FcyR-mediated depletion), anti-CSF1R (rat IgG2a, clone: AFS98; with competent FcyR-mediated depletion), and anti-CTLA-4 (murine IgG2a, clone: 12C11; with competent FcγR-mediated depletion) were used. Antibody treatment was administered via intraperitoneal (ip) injection at a dose of 10 mg/kg body weight for 3 doses every 4 days for CXCR2, 50 mg/kg body weight every 3 days for the duration of the experiment for CSF1R, and/or10 mg/kg body weight for 3 doses every 3 days for CTLA-4. Mouse IgG1 (clone: 4F7), rat IgG2a (clone: 2A3), and mouse IgG2a (clone: 4C6) were used as isotype controls. Anti-CXCR2 and anti-CSF1R treatments started 7 days before ADT; while anti-CTLA-4 treatment was started either 3 or 12 days before ADT (400 mm³ vs. 200 mm³, respectively).

### Flow Cytometry

Single-cell suspensions from prostate tumor and tissues were prepared using the Mouse Tumor Dissociation Kit according to the manufacturer's recommendations (Miltenyi). Single-cell suspensions of tumor-draining lymph nodes (TDLNs) and spleens were homogenized mechanically with the back of a syringe. Cells were Fc-blocked with purified rat anti-mouse CD16/CD32 (Clone: 2.4 G2, Becton Dickinson BD) for 15 minutes at RT. Dead cells were discriminated using the LIVE/DEAD (L/D) fixable viability dye eFluor 506 or near-IR dead cell stain kit (Thermo Fisher) and samples were stained for the extracellular and intracellular markers. The following antibodies were used: CD45 (30E-11), CD45.2 (104), CD24 (M1/69), CD49f (GOH3), Ly6C (HK1.4), Ly6G (1A8), Gr1 (RB6-8C5), CD11b (M1/70), F4/80 (BM8), MHCII (2G9), PD-L1 (10F.9G2), TCR13 (H57-597), CD4 (RM4-5), CD8 (53-6.7), CD44 (IM7), CD62L (MEL-14), CD25 (PC61), Ki67 (16A8), IFN-y (XMG1.2), TNF-a (MP6-XT22), IL-2 (JES6-5H4), GZβ (GB11), CXCR2 (242216), and IL-23 (FC23CPG). For intracellular staining, cells were fixed and permeabilized by using BD Perm/Wash (BD Biosciences) at room temperature for 45 minutes. For intracellular cytokine staining, cells were stimulated with PMA (50 ng/ml) and ionomycin (500 ng/ml) for 4 hours in the presence of protein transport inhibitor cocktail (eBiosciences). Gates of cytokines were determined by fluorescence minus one (FMO) controls. Staining was visualized by fluorescenceactivated cell sorting (FACS) analysis using a BD FACSCelesta™ (BD Biosciences) and analyzed using FlowJo® (Flowjo LLC). Prostate luminal epithelial cells are defined as CD45⁻CD11b⁻F4/80⁻CD24⁺CD49f^{int}GFP⁺, and prostate epithelial tumor cells are defined as CD45⁻CD11b⁻F4/80⁻mCherry⁺. Tumor associated macrophages (TAMs) are referred to as CD45⁺CD11b⁺F4/80⁺, inflammatory TAMs as CD45⁺CD11b⁺F4/80⁺Ly6C⁺MHCII⁻, immature TAMs as CD45⁺CD11b⁺F4/80⁺Ly6C⁺MHCII⁺, MHCII^{hi} TAMs as CD45⁺CD11b⁺F4/80⁺Ly6C⁻MHCII⁺, MHCII^{low} TAMs as CD45⁺CD11b⁺F4/80⁺Ly6C⁻MHCII⁻. PMN-MDSCs are defined as CD45⁺CD11b⁺Ly6C⁺Ly6G⁺. CD4 T cells as CD45⁺TCR6⁺CD4⁺, regulatory T cells as CD45⁺TCR6⁺CD4⁺CD25⁺, CD8 T cells as CD45⁺TCRβ⁺CD8⁺, polyfunctional CD8 T Cells as CD45⁺TCRβ⁺CD8⁺INFγ⁺TNFβ⁺Gzβ⁺, and memory CD8 T cells as CD45⁺TCRβ⁺CD8⁺CD44⁺CD62L⁻. 123Count eBeads counting beads (Thermo Fisher) were used to normalize the numbers of PMN-MDSCs in migration/chemotaxis experiments.

### Protein Quantification

Tumors collected at different treatment time points were minced, lysed in CelLytic MT (Sigma) containing halt protease and phosphatase inhibitor (Thermo Fisher) in a 1:100 ratio, and incubated on ice for 30 minutes with intermittent vortexing. Tumor lysates were assayed for raw protein concentration with Coomassie assay (Bio-Rad). IL-8 and Cxcl15 were analyzed by ELISA kits following the manufacturer's instructions (BD Bioscience and R&D Systems, respectively).

### Immunohistochemical Staining (IHC)

Tumor and tissue samples were fixed with either 10% formalin (Fisher Scientific, Pittsburgh, Pa.) or zinc fixative (BD) for 24 hours before paraffin embedding and sectioning. Sections were stained with hematoxylin and eosin (H&E), and antibodies against mouse Ly6G (1A8; BD Pharmingen) and F4/80 (BM8; eBioscience). Staining was performed by the Molecular Pathology core of the Herbert Irving Comprehensive Cancer Center at Columbia University. All images were acquired on a Leica SCN 400 system with high throughput 384 slide autoloader (SL801) and a 40× objective; files were processed with Aperio ImageScope v12.3.1.6002.

### RNA In Situ Hybridization (RISH) and Immunohistochemistry

Manual fluorescent RNAScope was performed on formalin-fixed and zinc-fixed paraffin embedded sections using company protocols. Briefly, sections were cut at 5 μm, air dried overnight, baked at 60° C. for 1 hrs, dewaxed and air-dried before pre-treatments. RNAScope Cxcl15, 3-plex positive control probes (Polr2a, Ppib, Ubc) and 3-plex negative control probes (DapB of Bacillus subtilis strain) from Advanced Cell Diagnostics (ACD) were used in this study. Detection of specific probe binding sites was with RNAScope Multiplex Fluorescent Reagent Kit v2 Reagent kit from ACD following the manufacture's instructions. Tyramide CF568 (Biotium) was used to visualize RISH signal.

For a more precise identification of cells expressing Cxcl15, the RISH was coupled to immunohistochemistry of PanCK (Poly; Dako) and CD45 (30-F11; BD Biosciences). Immediately after RISH detection, samples were permeabilized with 0.2% TBS-Tween 20 for 10 min at RT, and then blocked with 2.5% of normal goat serum (Vector) for 30 min at RT. Primary antibody for PanCK was diluted 1/400 in renaissance background reducing diluent (Biocare Medical) and they were incubated overnight at 4° C. After washing off the primary antibody, the slides were incubated 15 min at RT horseradish peroxidase (HRP) secondary antibody (Vector). Tyramide CF640R (Biotium) was used to visualize PanCK staining. In some cases, CD45 staining was also performed. For this, HRP signal was developed by a 30 min incubation at RT with PeroxAbolish (Biocare Medical) and then blocked with 2.5% of normal goat serum (Vector) for 30min at RT. Primary antibody for CD45 was diluted 1/50 in renaissance background reducing diluent (Biocare Medical) and they were incubated 1.5 hrs at RT. After washing off the primary antibody, the slides were incubated 15 min at RT HRP secondary antibody (Vector). Tyramide CF488A (Biotium) was used to visualize CD45 staining. All images were acquired on a Nikon A1RMP confocal microscope using a 60× objective. Comparisons of ISH-IHC results were performed using ImageJ.

### Whole Genome Expression Profiling and Analysis

MCRedAL tumor were harvested when their tumor volume reached ˜500 mm³ (CS group), and 7 days after chemical castration (ADT). MCRedAL cells were isolated based on their mCherry⁺ CD45⁻ F4/80⁻ CD11b⁻ expression by flow sorting on a DakoCytomation MoFlo. RNA was extracted using Trizol LS (Invitrogen) and treated with DNAse-I using RNA clean & Concentrator (Zymo Research). The analysis was performed using Affymetrix Mouse Clariom D (MTA 1.0) array according to the manufacturer's instructions. Resulting CEL files were analyzed in Affymetrix Expression Console (v. 1.4) using the SST-RMA method, and all samples passed the quality control. Log2 probe intensities were extracted from CEL (signal intensity) files and normalized using RMA quantile normalization, then further analyzed using Partek Genomics Suite v6.6. Illustrations (volcano plots, heatmaps, and histograms) were generated using TIBCO Spotfire DecisionSite with Functional Genomics. Gene set enrichment analysis (GSEA) of differently expressed genes was performed using the hallmark gene sets Molecular Signature Database (MSigDB).

### Nanostring

RNA extraction was performed using the TRIzol LS reagent (Thermo Fisher) as per manufacturer's instructions. For NanoString analysis, the nCounter mouse PanCancer Immune Profiling panel was employed using the nCounter Analysis System (both NanoString, Seattle, Wash.). Analysis was conducted using nSolver software (NanoString). Heatmap analysis were performed using The R Project for Statistical Computing (https://www.r-project.org/).

### Pairwise Alignment

The homology of the murine chemokines Cxcl1, Cxcl2, Cxcl5, Cxcl15, Cxcl12, and Cxcl17 to human IL-8 was evaluated using BLASTP 2.9.0+ (https://blast.ncbi.nlm.nih.gov/Blast.cgi?PAGE=Proteins). Proteins were considered homologous if they shared >30% amino acid identity. Expected values of <0.05 were consider statistically significant. The expected value includes an inherent Bonferroni correction.

Chromatin Immunoprecipitation Assay https://www.ncbi.nlm.nih/gov/geo/query/acc.egi?acc=GSE83860 which contains ChIP-Seq data acquired with androgen receptor (AR) and nuclear factor NF-kappa-B p65 subunit (p65) specific antibodies on cell lysates from LNCaP cells cultured under the following treatments: DMSO, DHT, and TNFα. For each treatment the dataset contains two ChIP-Seq replicates pulled down using the AR and p65 antibodies. ChIP-Seq data were aligned to the hg38 reference version using the subread package, and then the BAM files were sorted and indexed using SAMtools. Loci with significant differential binding (FDR=0.05) of pulled-down proteins to DNA were identified using the csaw package for ChIP-Seq analysis, closely following Lun and Smyth's script⁷. ChIP-Seq visualization was performed using the Integrative Genomics Viewer (IGV) from the Broad Institute (http://software.broadinstitute.org/software/igv.).

### ChIP-qRT-PCR

Chromatin immunoprecipitation was performed. In brief, LNCaP cells were washed with serum-free media and then grown in media containing 10% charcoal stripped FBS for 48 hours. Cells were treated with 100 nM DHT or vehicle for 8 hours. DNA was cross-linked with 1% formaldehyde in PBS for 10 minutes and crosslinking was quenched by addition of 0.125 M glycine. Fixed cells were then lysed in lysis buffer (1% SDS, 5 mM EDTA, 50 mM Tris HCl, pH8.1) and sonicated to a fragment size of 200-600 bp using a Covaris water bath sonicator (Woburn, Mass.). Sheared chromatin was then incubated with primary antibodies (AR [06-680, Millipore], H3K4me3 [ab8580, Abcam], phospho-SerS RNA polymerase 2 [ab5131, Abcam], RNA polymerase 2 [4H8, Cell Signaling Technologies] or control IgG [Cell Signaling Technologies]) overnight at 4° C. Complexes were immobilized on Dynabeads (Thermo Fisher) by incubating for 4 hours at 4° C. Beads were sequentially washed with TSEI (0.1% SDS, 1% Triton X-100, 2 mM EDTA, 20 mM Tris HCl, pH 8.1, 150 mM NaCl), TSEII (0.1% SDS, 1% Triton X-100, 2 mM EDTA, 20 mM Tris HCl, pH 8.1, 500 mM NaCl) and TSEIII (0.25 M LiCl, 1% NP-40, 1% deoxycholate, 1 mM EDTA, 10 mM Tris HCl, pH 8.1). DNA was eluted with IP Elution buffer (1% SDS, 0.1M NaHCO₃, proteinase K) and incubated at 56° C. for 15 minutes. Enriched DNA libraries were analyzed using primers specific to IL-8 locus: Forward: 5′ AGCTGCAGAAATCAGGAAGG 3′ (SEQ ID NO: 1) and Reverse: 5′ TATAAAAAGCCACCGGAGCA 3′ (SEQ ID NO: 2) using quantitative (q) RT-PCR. Data is shown as relative enrichment normalized to input DNA.

### Quantitative (q) RT-PCR

Total RNA was extracted using Trizol (Ambion). cDNA was prepared from total RNA preps using the RNA to cDNA EcoDry Premix (Clontech). Real-time assays were conducted using TaqMan real-time probes (Applied Biosystems). AA CT method was used for relative gene expression. Expression of the target gene was normalized to the reference gene (18S) and the mean expression level of the control group. LCM samples were normalized to 18S, TBP, and GAPDH reference genes.

### Laser Capture Microscopy (LCM)

Formalin fixed-paraffin embedded radical prostatectomy specimens, from patients enrolled in a neoadjuvant clinical trial (NCT01696877) who received 240 mg (SQ) of degarelix and matched control cases (patients that did not receive any hormone therapy), were sectioned at a thickness of 8 pm and transferred onto PEN membrane glass slides (Leica). Sections were deparaffinized, hydrated and stained with hematoxylin prior to microdissection. Individual cancer cells and cancer cell clusters were microdissected by a trained pathologist using a LMD 7000 laser capture microscope (Leica). RNA was recovered from the microdisseceted material using the RNeasy FFPE kit (Qiagen). Quantitative RT-PCR was performed as described above. For the analysis, a Mann-Whitney U test was performed.

### IL-8 and Cxcl15 CRISPR/Cas9 Knock Outs

The 20 bp long gRNA, designed using Deskgen online software, for targeting IL-8 and Cxcl15 in exon 3 (5′-TTCAGTGTAAAGCTTTCTGA-3′ (SEQ ID NO: 3) and 5′-ACAGAGCAGTCCCAAAAAAT-3′ (SEQ ID NO: 4), respectively) were incorporated into two complementary 100-mer oligonucleotides and cloned into a gRNA containing plasmid containing the (NeoR/KanR) cassette (Addgene #41824). The human codon optimized pCAGGS-Cas9-mCherry was used for gene-editing experiments (a gift from Stem Cell Core Facility at Columbia University). gRNA and Cas9 containing plasmids were introduced to prostate epithelial cells using the basic nucleofeofector kit (Amaxa, Lonza) following the manufacture's instructions for primary mammalian epithelial cell (program W001). Successfully transfected cells were selected by culturing in the presence of 400 μg/ml of neomycin sulfate analog (G418; Sigma), and isolated based on their mCherry expression 24 hours after transfection. Knock out clones were screened for IL-8 and Cxcl15 expression by ELISA and gene-editing confirmed by PCR-sequencing using primers ˜200 bp away from the cut site

(IL-8 F: 5′-TTTGGACTTAGACTTTATGCCTGAC-3 (SEQ ID NO: 5);
IL-8 R: 5′-TCCTGGGCAAACTATGTATGG-3 (SEQ ID NO: 6);
Cxcl15 F: 5′-GCTAGGCACACTGATATGTGTTAAA-3 (SEQ ID NO: 7);
Cxcl15 R: 5′-ACATTTGGGGATGCTACTGG-3 (SEQ ID NO: 8)).

### Migration/Chemotaxis Assay

Cells and supernatants used in this assay were resuspended in culture media containing 0.5% or 2.5% FBS. Transwell plates of 3-mm pore size were coated with Fibronectin (Corning Costar) and loaded with 500 ml of medium or with different cell supernatants in triplicates (lower chamber). Cells were resuspended at 2×10⁷ cells/ml, and 200 ml of this suspension was placed in each of the inserts (upper chamber). After 2.5 hours of incubation at 37° C. and 5% CO₂, inserts were removed and 10,000 beads (Thermo Fisher) were added to each well. In some cases, either isotype or anti-CXCR2 (200 μg/ml) were added at the beginning of the experiment. The cells in the lower chamber were collected along with the starting cell population, stained with L/D, CD11b, Ly6C, and Ly6G and evaluated by flow cytometry in a BD FACSCelesta™ (BD Biosciences). The ratio of beads to cells was determined, allowing calculation of the number of cells that had migrated to the bottom well. In vivo, LD-PMN-MDSCs were collected as described below from splenocytes of CR-Myc-Cap tumor bearing mice and labeled with DiD (DiIC18(5) or 1,1′-Dioctadecyl-3,3,3′,3′-Tetramethylindodicarbocyanine, 4-Chlorobenzenesulfonate Salt; Invitrogen), a lipophilic membrane dye, as described previously. DiD⁺ LD-PMN-MDSCs were adoptively transferred into FVB/NJ recipient 8-week male mice and their ability to migrate in response to 200 ng of recombinant Cxcl15 was evaluated 4 hours after injection. Beads were also used to calculate absolute numbers of Ly6G⁺ PMNs and DiD⁺ LD-PMN-MDSCs in vivo.

### PMN-MDSC Enrichment

Animals were sacrificed and spleens were collected. After dissociating cell clumps, the cell suspension was centrifuged (740 g, 10 minutes, RT) and resuspended in 1 ml HBSS-EDTA containing 0.5% BSA. Cells were then resuspended in 50% Percoll solution and treated on a three-layer Percoll gradient (55%, 72%, and 81%) at (1500 g, 30 minutes, 10° C. without break). LD-PMN-MDSCs were collected from the 50-55% and 55-72% interfaces. Red blood cells (RBCs) were eliminated with RBC lysis solution (Miltenyi).

### In Vitro Suppression Assays

PMN-MDSCs were isolated from the spleen of CR-Myc-Cap-tumor bearing mice using the neutrophil isolation kit (Miltenyi) according to the manufacturer's instructions; greater than 95% enrichment was confirmed by flow cytometry. Unless otherwise indicated, a density gradient separation was performed prior to column purification. OT-I (CD45.2) transgenic splenocytes were mixed at a 1:10 ratio with sex-matched CD45.1 splenocytes. Splenocytes containing CD8 T responder cells were stained with CellTrace Violet (5 μM CTV; Thermo Fisher) and plated on a 96-well round-bottom plate at a density of 2×10⁵ cells per well. PMN-MDSCs cells were added at 2-fold dilutions starting from 2×10⁵ cells, in the presence of their cognate peptides (5 μM OVA) and incubated for 60 hours. Proliferation of CD8 T responder cells (gated as L/D⁻CD8⁺CTV⁺) was quantified by flow cytometry based on the dilution of Cell Trace Violet (CTV). Percent suppression (% Suppression) was calculated by the following formula: %

Suppression=[1−(% divided cells of the condition/ the average of % divided cells of T responder only conditions)]×100.

### Z-Score Analysis

IL-8 expression was evaluated in a publicly available data set (GSE8466)³⁷ using z-score values of quantile-normalized microarray transcripts from benign prostate biopsies. Z-score values were obtained by scaling the data for each gene in each patient to: (expression—mean expression across all genes)/(standard deviation of expression across all genes).

### Statistical Analysis

Statistical analysis was performed using Prism 7 (GraphPad). Unpaired two-tailed t-tests, Mann-Whitney U test, Tukey's multiple comparisons tests, or Wilcoxon rank sum tests were conducted and considered statistically significant at p-values 0.05 (*), 0.01 (**), 0.001 (***) and 0.0001 (****).

Although the methods of the invention have been described in the present disclosure by way of illustrative examples, it is to be understood that the invention is not limited thereto and that variations can be made as known by those skilled in the art without departing from the teachings of the invention defined by the appended claims.

What is claimed is:

1. A method of treating prostate cancer in a patient in need of such treatment which comprises administering to said patient in combination with androgen ablation a therapeutically effective amount of an IL-8 blocker selected from an IL-8 antagonist and an IL-8 receptor antagonist.

2. The method of claim 1 wherein the androgen ablation is selected from a bilateral orchiectomy and administration of a therapeutically effective androgen ablation amount of a compound selected from LHRH agonist (leuprolide, goserelin, triptorelin, or histrelin), an LHRH antagonist (degarelix, a CYP17 inhibitor, or abiraterone); and an anti-androgen (flutamide, bicalutamide, nilutamide, enzalutamide, or apalutamide), and a combination thereof.

3. The method of claim 2 wherein the androgen ablation is administration of an effective androgen ablation amount of a compound selected from LHRH agonist (leuprolide, goserelin, triptorelin, or histrelin), an LHRH antagonist (degarelix, a CYP17 inhibitor, or abiraterone); and an anti-androgen (flutamide, bicalutamide, nilutamide, enzalutamide, or apalutamide), and a combination thereof (ADT).

4. The method of claim 1 wherein the IL-8 antagonist or IL-8 receptor antagonist is selected from BMS-986253 (HuMax IL-8), ABX-IL8, HuMab 10F8, SCH527123/MK-7123, AZD5069, AZD5122, AZD8304, RIST4721, CCX832, a CX3CR1 antagonist, a CXCR1/2 monoclonal IL-8 blocker, a CXCR2 antagonist, CXCR2 biparatopic nanobodies, a CXCR2 monoclonal IL-8 blocker, DF1970, DF2726A, DF2156A, DF2162, DF2755A, repertaxin, reparixin, FX68, GSK1325756, GSK1325756H, SB225002, SB251353, SB332235, SB656933, KB03, MGTA145, PACG31P, PS291822 (navarixin), SX576, SX682, and human ELR+CXC chemokine blockers.

5. The method of claim 2 wherein the IL-8 antagonist, or IL-8 receptor antagonist is selected from BMS-986253 (HuMax IL-8), ABX-IL8, HuMab 10F8, SCH527123/MK-7123, AZD5069, AZD5122, AZD8304, RIST4721, CCX832, a CX3CR1 antagonist, a CXCR1/2 monoclonal IL-8 blocker, a CXCR2 antagonist, CXCR2 biparatopic nanobodies, a CXCR2 monoclonal IL-8 blocker, DF1970, DF2726A, DF2156A, DF2162, DF2755A, repertaxin, reparixin, FX68, GSK1325756, GSK1325756H, SB225002, SB251353, SB332235, SB656933, KB03, MGTA145, PACG31P, PS291822 (navarixin), SX576, SX682, and human ELR+CXC chemokine blockers.

6. The method of claim 5 wherein the androgen ablation compound is degarelix.

7. The method of claim 3 wherein the IL-8 blocker is administered to the patient before the ADT.

8. A method of treating prostate cancer in a patient in need of such treatment which comprises administering to said patient in combination with androgen ablation an amount of an IL-8 blocker selected from an IL-8 antagonist and an IL-8 receptor antagonist, the amount of said IL-8 blocker being effective to at least reduce the infiltration of suppressive immune cells into prostate tumor.

9. The method of claim 8 wherein the androgen ablation is selected from a bilateral orchiectomy and administration of an effective androgen ablation amount of a compound selected from LHRH agonist (leuprolide, goserelin, triptorelin, or histrelin), an LHRH antagonist (degarelix, a CYP17 inhibitor, or abiraterone); and an anti-androgen (flutamide, bicalutamide, nilutamide, enzalutamide, or apalutamide), and a combination thereof (ADT).

10. The method of claim 9 wherein the androgen ablation is ADT and the IL-8 blocker is administered before the ADT.

11. The method of claim 8 wherein the IL-8 antagonist or IL-8 receptor antagonist is selected from BMS-986253 (HuMax IL-8), ABX-IL8, HuMab 10F8, SCH527123/MK-7123, AZD5069, AZD5122, AZD8304, RIST4721, CCX832, a CX3CR1 antagonist, a CXCR1/2 monoclonal IL-8 blocker, a CXCR2 antagonist, CXCR2 biparatopic nanobodies, a CXCR2 monoclonal IL-8 blocker, DF1970, DF2726A, DF2156A, DF2162, DF2755A, repertaxin, reparixin, FX68, GSK1325756, GSK1325756H, SB225002, SB251353, SB332235, SB656933, KB03, MGTA145, PACG31P, PS291822 (navarixin), SX576, SX682, and human ELR+CXC chemokine blockers.

12. The method of claim 9 wherein the androgen ablation is ADT and the IL-8 antagonist or IL-8 receptor antagonist is selected from BMS-986253 (HuMax IL-8), ABX-IL8, HuMab 10F8, SCH527123/MK-7123, AZD5069, AZD5122, AZD8304, RIST4721, CCX832, a CX3CR1 antagonist, a CXCR1/2 monoclonal IL-8 blocker, a CXCR2 antagonist, CXCR2 biparatopic nanobodies, a CXCR2 monoclonal IL-8 blocker, DF1970, DF2726A, DF2156A, DF2162, DF2755A, repertaxin, reparixin, FX68, GSK1325756, GSK1325756H, SB225002, SB251353, SB332235, SB656933, KB03, MGTA145, PACG31P, PS291822 (navarixin), SX576, SX682, and human ELR+CXC chemokine blockers.

13. The method of claim 12 wherein the androgen ablation compound is degarelix.

14. The method of claim 9 wherein the androgen ablation is ADT and the IL-8 blocker is administered to the patient before the ADT.

15. The method of claim 1 which further comprises administration of a therapeutically effective amount of an immunotherapeutic agent selected from anti-PD-1, anti-PD-L1, anti-CTLA-4, anti TIM-3, anti-TGIT, anti-CD40, a TLR agonist, a STING agonist, bi-specific T cell engagers (BiTEs) and dual-affinity retargeting antibodies (DARTs), chimeric antigen receptors (CARs) T cells, and an anti-cancer vaccine selected from Sipuleucel-T, PSA-TRICOM, AVAX Tech vaccines, Prostvac-VF, and a listeria-based vaccines selected from live attenuated double-deleted (LADD) and ADXS031-142.

16. The method of claim 3 which further comprises administration of a therapeutically effective amount of an immunotherapeutic agent selected from anti-PD-1, anti-PD-L1, anti-CTLA-4, anti TIM-3, anti-TGIT, anti-CD40, a TLR agonist, a STING agonist, bi-specific T cell engagers (BiTEs) and dual-affinity retargeting antibodies (DARTs), chimeric antigen receptors (CARs) T cells, and an anti-cancer vaccine selected from Sipuleucel-T, PSA-TRICOM, AVAX Tech vaccines, Prostvac-VF, and a listeria-based vaccine selected from live attenuated double-deleted (LADD) and ADXS031-142.

17. The method of claim 5 which further comprises administration of a therapeutically effective amount of an immunotherapeutic agent selected from anti-PD-1, anti-PD-L1, anti-CTLA-4, anti TIM-3, anti-TGIT, anti-CD40, a TLR agonist, a STING agonist, bi-specific T cell engagers (BiTEs) and dual-affinity retargeting antibodies (DARTs), chimeric antigen receptors (CARs) T cells, and an anti-cancer vaccine selected from Sipuleucel-T, PSA-TRICOM, AVAX Tech vaccines, Prostvac-VF, and a listeria-based vaccines selected from live attenuated double-deleted (LADD) and ADXS031-142.

18. The method of claim 7 which further comprises administration of a therapeutically effective amount of an immunotherapeutic agent selected from anti-PD-1, anti-PD-L1, anti-CTLA-4, anti TIM-3, anti-TGIT, anti-CD40, a TLR agonist, a STING agonist, bi-specific T cell engagers (BiTEs) and dual-affinity retargeting antibodies (DARTs), chimeric antigen receptors (CARs) T cells, and an anti-cancer vaccine selected from Sipuleucel-T, PSA-TRICOM, AVAX Tech vaccines, Prostvac-VF, and a listeria-based vaccine selected from live attenuated double-deleted (LADD) and ADXS031-142.

19. The method of claim 8 which further comprises administration of a therapeutically effective amount of an immunotherapeutic agent selected from anti-PD-1, anti-PD-L1, anti-CTLA-4, anti TIM-3, anti-TGIT, anti-CD40, a TLR agonist, a STING agonist, bi-specific T cell engagers (BiTEs) and dual-affinity retargeting antibodies (DARTs), chimeric antigen receptors (CARs) T cells, and an anti-cancer vaccine selected fromSipuleucel-T, PSA-TRICOM, AVAX Tech vaccines, Prostvac-VF, and a listeria-based vaccine selected from live attenuated double-deleted (LADD) and ADXS031-142.

20. The method of claim 10 which further comprises administration of a therapeutically effective amount of an immunotherapeutic agent selected from anti-PD-1, anti-PD-L1, anti-CTLA-4, anti TIM-3, anti-TGIT, anti-CD40, a TLR agonist, a STING agonist, bi-specific T cell engagers (BiTEs) and dual-affinity retargeting antibodies (DARTs), chimeric antigen receptors (CARs) T cells, and an anti-cancer vaccine selected from Sipuleucel-T, PSA-TRICOM, AVAX Tech vaccines, Prostvac-VF, and a listeria-based vaccine selected from live attenuated double-deleted (LADD) and ADXS031-142.

21. The method of claim 12 which further comprises administration of a therapeutically effective amount of an immunotherapeutic agent selected from anti-PD-1, anti-PD-L1, anti-CTLA-4, anti TIM-3, anti-TGIT, anti-CD40, a TLR agonist, a STING agonist, bi-specific T cell engagers (BiTEs) and dual-affinity retargeting antibodies (DARTs), chimeric antigen receptors (CARs) T cells, and an anti-cancer vaccine selected from Sipuleucel-T, PSA-TRICOM, AVAX Tech vaccines, Prostvac-VF, and a listeria-based vaccine selected from live attenuated double-deleted (LADD) and ADXS031-142.

22. The method of claim 10 which further comprises administration of a therapeutically effective amount of an immunotherapeutic agent selected from anti-PD-1, anti-PD-L1, anti-CTLA-4, anti TIM-3, anti-TGIT, anti-CD40, a TLR agonist, a STING agonist, bi-specific T cell engagers (BiTEs) and dual-affinity retargeting antibodies (DARTs), chimeric antigen receptors (CARs) T cells, and an anti-cancer vaccine selected from Sipuleucel-T, PSA-TRICOM, AVAX Tech vaccines, Prostvac-VF, and a listeria-based vaccine selected from live attenuated double-deleted (LADD) and ADXS031-142.

23. The method of claim 15 wherein the androgen ablation is ADT and the immunotherapeutic agent is administered to the patient before the ADT.

24. The method of claim 3 wherein the immunotherapeutic agent is administered to the patient before the ADT.

25. The method of claim 15 wherein the IL-8 blocker is anti-CXR2 and the immunotherapy agent is anti-CTLA-4.
